(19) United States
(12) Patent Application Publication
(10) Pub. No.: US 2023/0028590 A1
(43) Pub. Date: Jan. 26, 2023

(54) ALS TREATMENT METHOD, ALS PHARMACEUTICAL COMPOSITION, AND ALS DIAGNOSTIC METHOD

(71) Applicants: MITSUBISHI TANABE PHARMA CORPORATION, Osaka (JP); MASSACHUSETTS INSTITUTE OF TECHNOLOGY, Cambridge, MA (US)

(72) Inventors: Manolis KELLIS, Cambridge, MA (US); Sergio Sebastian PINEDA, Cambridge, MA (US); Myriam HEIMAN, Cambridge, MA (US); Makoto TAMURA, Cambridge, MA (US)

(73) Assignees: MITSUBISHI TANABE PHARMA CORPORATION, Osaka (JP); MASSACHUSETTS INSTITUTE OF TECHNOLOGY, Cambridge, MA (US)

(21) Appl. No.: 17/810,277

(22) Filed: Jun. 30, 2022

Related U.S. Application Data
(60) Provisional application No. 63/216,789, filed on Jun. 30, 2021.
(60) Provisional application No. 63/263,248, filed on Oct. 29, 2021.

(51) Int. Cl. C12N 15/113; A61P 21/00

(52) U.S. Cl. CPC C12N 15/113; A61P 21/00

(57) ABSTRACT

An ALS treatment method includes administering to an ALS patient an inhibitor for a target A or a promoter for a target B. The target A is one or more genes selected from the genes in Table 1-1 or a protein encoded by the gene, and the inhibitor for the target A is a substance that inhibits expression of the gene, or a substance that inhibits a function of the protein encoded by the gene. The target B is one or more genes selected from the genes in Table 1-2 or a protein encoded by the gene, and the promoter for the target B is a substance that promotes the expression of the gene, or a substance that promotes a function of the protein encoded by the gene.

### CROSS-REFERENCE TO RELATED APPLICATIONS

The present application is based upon and claims the benefits of priority to U.S. Provisional Patent Applications No. 63/216,789, filed Jun. 30, 2021 and No. 63/263,248, filed Oct. 29, 2021. The entire contents of these applications are incorporated herein by reference.

### BACKGROUND OF THE INVENTION

### Field of the Invention

The present invention relates to an ALS treatment method, an ALS pharmaceutical composition, and an ALS diagnostic method.

### Description of Background Art

Amyotrophic lateral sclerosis (ALS) is a progressive neurodegenerative disease that causes muscle atrophy and muscle weakness due to disorders of motor neurons, and is designated as an intractable disease. As ALS progresses, paralysis of the limbs, respiratory paralysis, dysphagia, and the like occur, and within a few years after the onset, it becomes difficult for the patient to move his/her body on his/her own will, and it is also necessary to wear a respirator or the like.

Autosomal dominant inheritance mutations are known in about 10% of ALS, but no clear inheritance mutations have been reported in the remaining 90% of ALS, and their etiology has not yet been clarified. Various studies have been conducted on the treatment of ALS, but no effective treatment method leading to a radical treatment has been established. Therefore, it is demanded to elucidate the more relevant etiology of ALS and to establish a treatment method based on it.

### SUMMARY OF THE INVENTION

According to one aspect of the present invention, a method for treating amyotrophic lateral sclerosis includes administering an inhibitor for a target A or a promotor for a target B to a patient in need thereof. The target A is one or more genes selected from genes in Table 1-1 or a protein encoded thereby, the inhibitor for the target A inhibits expression of the gene or a function of the protein encoded by the gene, the target B is one or more genes selected from genes in Table 1-2 or a protein encoded thereby, and the promotor for the target B promotes expression of the gene or a function of the protein encoded by the gene.

According to another aspect of the present invention, a pharmaceutical composition for treating ALS includes an inhibitor for a target A or a promotor for a target B. The target A is one or more genes selected from genes in Table 1-1 or a protein encoded thereby, the inhibitor for the target A inhibits expression of the gene or a function of the protein encoded by the gene, the target B is one or more genes selected from genes in Table 1-2 or a protein encoded thereby, and the promotor for the target B promotes expression of the gene or a function of the protein encoded by the gene.

According to yet another aspect of the present invention, a method for treating ALS includes acquiring an expressed amount of a target A or a target B in a biological sample of a subject, and determining that the subject is in need of treating ALS when the expression amount of the target A of the subject is higher than an expression amount of the target A of a healthy subject and/or when the expression amount of the target B of the subject is lower than an expression amount of the target B of the healthy subject. The target A is one or more genes selected from genes in Table 1-1 or a protein encoded thereby, and the target B is one or more genes selected from genes in Table 1-2 or a protein encoded thereby.

According to still another aspect of the present invention, a method for treating amyotrophic lateral sclerosis includes administering an inhibitor or a promotor for a gene expression or a protein function encoded by a target gene to a patient in need thereof. The target gene is related to receptor diffusion trapping, the inhibitor inhibits the gene expression or the protein function, and the promotor promotes the gene expression or the protein function.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows the genes of the target A (Table 1-1) and the genes of the target B (Table 1-2);

FIG. 2 shows characterization of ALS and FTLD implicated cell populations and includes FIG. 2A showing representative images showing indirect immunofluorescent labeling of Betz cell markers Mu-Crystallin (CRYM) and POU3F1 in pathologically normal tissue (Scale bar=20 μm), FIG. 2B (2B-1. and 2B-12.) showing differential expression of top 50 marker genes of annotated subtypes corresponding to previously described and newly identified differentially vulnerable cell types in ALS and FTLD, and right-most column showing expression of markers in VENs of the dorsolateral PFC (Top 25 markers of each subtype labeled on the left), and FIG. 2c showing violin plots showing single-cell-level expression of VEN marker genes from Hodge et al. (2020) in annotated excitatory subtypes of MCX and VENs of PFC (Both Betz cell clusters in MCX (MCX Ex L5b BCL11B) and the VEN cluster in PFC (PFC EX L5b POU3F1) exhibit high expression of nearly every identified VEN marker, and dots denote population mean. MCX: motor cortex; PFC: prefrontal cortex);

FIG. 3 shows differential gene expression analysis and includes FIG. 3A showing absolute number of DEGs detected per cell type per disease group (absolute log 2-fold change Z-score>1, FDR-adjusted p<0.005), FIG. 3B showing disease score (transcriptomic distance from pathologically normal) of each cell type per disease group. Left: Absolute distance (Right: Column-wise Z-score of distances), FIG. 3C showing overlap of detected pyramidal tract upper motor neuron408 (Betz cell) group DEGs across disease groups; and FIG. 3D (3D-1 to 3D-6) showing top up and down regulated pan-phenotypic and phenotype specific DEGs (FDR-adjusted p<0.005) (ns: Not statistically significant; NA: absent in data set);

FIG. 4 shows WGCNA analysis of Ex UMN PT (Betz) cluster in ALS and FTLD and includes FIG. 4A showing dendrogram showing result of unsupervised hierarchical clustering of modules identified by WGCNA, FIG. 4B showing Pearson correlation of module eigengenes with disease groups (Modules significantly correlated with ALS and/or FTLD are shown, with the number of hub genes displayed on the right of each module's name), and FIG. 4C showing Significant KEGG pathways enriched in hub genes of “blue”, “brown”, “steelblue”, and “darkorange2” modules (Numbers in each circle represent the number of hub genes in each pathway from the corresponding module); and

FIG. 5 shows POU3F1 is enriched in CRYM+ cells and displays altered subcellular localization in ALS and FTLD patient tissues (Representative images showing indirect immunofluorescent labeling of POU3F1 and TDP-43 (TARDBP) in Betz cells of pathologically normal (PN), sporadic ALS (sALS), C9orf72-associated ALS (c9ALS), sporadic FTLD (sFTLD), and C9orf72-associated FTLD (c9FTLD) patient tissues. Arrows indicate select regions displaying colocalized, punctate morphology of POU3F1 and TDP-43 fluorescent signals. Scale bar=10 μm).

### DETAILED DESCRIPTION OF THE EMBODIMENTS

Embodiments will now be described with reference to the accompanying drawings, wherein like reference numerals designate corresponding or identical elements throughout the various drawings.

In the ALS treatment method of the present invention, for example, the expression level of the target A or the target B is acquired from the biological sample of the patient.

When the expression level of the target A of the patient is higher than an expression level of the target A of a healthy subject, an inhibitor for the target A is administered to the patient. For example, in one embodiment, a subject is a person.

When the expression level of the target B of the patient is lower than an expression level of the target B of a healthy subject, a promoter for the target B is administered to the patient.

In the ALS treatment method of the present invention, for example, the patient has no ALS-related mutation in at least one gene selected from a group consisting of a C9orf72 gene, a SOD1 gene, a TBK1 gene, a TARDBP gene, a FUS gene and a NEK1 gene, the target A is a target A1, the target A1 is at least one gene selected from a group consisting of genes in Table 2-1 or a protein encoded by the gene, the target B is a target B1, and the target B1 is at least one gene selected from a group consisting of genes in Table 2-2 or a protein encoded by the gene.

In the ALS treatment method of the present invention, for example, the patient has no ALS-related mutation in the C9orf72 gene.

In the ALS treatment method of the present invention, for example, the patient has an ALS-related mutation in the C9orf72 gene, the target A is a target A2, the target A2 is at least one gene selected from a group consisting of genes in Table 3-1 or a protein encoded by the gene, the target B is a target B2, and the target B2 is at least one gene selected from a group consisting of genes in Table 3-2 or a protein encoded by the gene.

In the ALS treatment method of the present invention, for example, the target A is a target A3, the target A3 is at least one gene selected from a group consisting of genes in Table 4-1 or a protein encoded by the gene, the target B is a target B3, and the target B3 is at least one gene selected from a group consisting of genes in Table 4-2 or a protein encoded by the gene.

In the ALS treatment method of the present invention, for example, the biological sample is a sample collected from the brain, and, as a specific example, is a cerebrospinal fluid.

In the ALS treatment method of the present invention, for example, the inhibitor for the target A is an inhibitor that inhibits expression of the gene, a substance that inhibits transcription of the gene, or a substance that inhibits translation from the gene.

In the ALS treatment method of the present invention, for example, the substance that inhibits the expression of the gene is at least one selected from a group consisting of an interfering nucleic acid, an antisense, and a ribozyme, and vectors expressing these substances.

In the ALS treatment method of the present invention, for example, the inhibitor for the target A is a substance that inhibits a function of the protein encoded by the gene, and is an antibody or an antigen-binding fragment against the protein, or an aptamer against the protein.

In the ALS treatment method of the present invention, for example, the promoter for the target B is a substance that promotes expression of the gene, and is a vector that expresses the gene.

In the ALS treatment method of the present invention, for example, the promoter for the target B is a substance that promotes a function of the protein encoded by the gene, and is the protein itself.

In the ALS treatment method of the present invention, for example, the receptor diffusion trapping is postsynaptic neurotransmitter receptor diffusion trapping or neurotransmitter receptor diffusion trapping.

In the ALS pharmaceutical composition of the present invention, for example, a subject to be administered is a patient who has no ALS-related mutation in at least one gene selected from a group consisting of a C9orf72 gene, a SOD1 gene, a TBK1 gene, a TARDBP gene, a FUS gene and a NEK1 gene, the target A is a target A1, the target A1 is at least one gene selected from a group consisting of genes in Table 2-1 or a protein encoded by the gene, the target B is a target B1, and the target B1 is at least one gene selected from a group consisting of genes in Table 2-2 or a protein encoded by the gene.

In the ALS pharmaceutical composition of the present invention, for example, a subject to be administered has an ALS-related mutation in the C9orf72 gene, the target A is a target A2, the target A2 is at least one gene selected from a group consisting of genes in Table 3-1 or a protein encoded by the gene, the target B is a target B2, and the target B2 is at least one gene selected from a group consisting of genes in Table 3-2 or a protein encoded by the gene.

In the ALS pharmaceutical composition of the present invention, for example, the target A is a target A3, the target A3 is at least one gene selected from a group consisting of genes in Table 4-1 or a protein encoded by the gene, the target B is a target B3, and the target B3 is at least one gene selected from a group consisting of genes in Table 4-2 or a protein encoded by the gene.

The ALS pharmaceutical composition of the present invention is, for example, a composition for injection or infusion.

In the ALS pharmaceutical composition of the present invention, for example, the inhibitor for the target A is an inhibitor that inhibits expression of the gene, a substance that inhibits transcription of the gene, or a substance that inhibits translation from the gene.

In the ALS pharmaceutical composition of the present invention, for example, the substance that inhibits the expression of the gene is at least one selected from a group consisting of an interfering nucleic acid, an antisense, and a ribozyme, and vectors expressing these substances.

In the ALS pharmaceutical composition of the present invention, for example, the inhibitor for the target A is a substance that inhibits a function of the protein encoded by the gene, and is an antibody or an antigen-binding fragment against the protein, or an aptamer against the protein.

In the ALS pharmaceutical composition of the present invention, for example, the promoter for the target B is a substance that promotes expression of the gene, and is a vector that expresses the gene.

In the ALS pharmaceutical composition of the present invention, for example, the promoter for the target B is a substance that promotes a function of the protein encoded by the gene, and is the protein itself.

In the ALS diagnostic method of the present invention, for example, expression of a target A4 of the target A or expression of a target B4 of the target B is acquired from a biological sample of the subject, the target A4 is at least one gene selected from a group consisting of genes in Table 5-1 or a protein encoded by the gene, when an expression level of the target A4 of the subject is higher than an expression level of the target A4 of a healthy subject, it is determined that the subject is suffering from ALS and is of a type having no ALS-related mutation in a C9orf72 gene, the target B4 is at least one gene selected from a group consisting of genes in Table 5-2 or a protein encoded by the gene, and when an expression level of the target B4 of the subject is lower than an expression level of the target B4 of a healthy subject, it is determined that the subject is suffering from ALS and is of a type having no ALS-related mutation in a C9orf72 gene.

In the ALS diagnostic method of the present invention, for example, expression of a target A5 of the target A or expression of a target B5 of the target B is acquired from a biological sample of the subject, the target A5 is at least one gene selected from a group consisting of genes in Table 6-1 or a protein encoded by the gene, when an expression level of the target A5 of the subject is higher than an expression level of the target A5 of a healthy subject, it is determined that the subject is suffering from ALS and is of a type having an ALS-related mutation in a C9orf72 gene, the target B5 is at least one gene selected from a group consisting of genes in Table 6-2 or a protein encoded by the gene, and when an expression level of the target B5 of the subject is lower than an expression level of the target B5 of a healthy subject, it is determined that the subject is suffering from ALS and is of a type having an ALS-related mutation in a C9orf72 gene.

In the ALS diagnostic method of the present invention, for example, the biological sample is a sample derived from the brain, and, as a specific example, is a cerebrospinal fluid.

In the following, the present invention is described with reference to specific examples.

In the present specification, treatment has a broad meaning and, for example, includes treatment in a narrow sense such as suppression of progression, improvement (alleviation), and radical cure, and includes meaning of prevention such as prevention of onset, and prevention of recurrence. In the present invention, for example, any one of these may be used as a purpose, or two or more of these may be used as a purpose.

### ALS Treatment Method

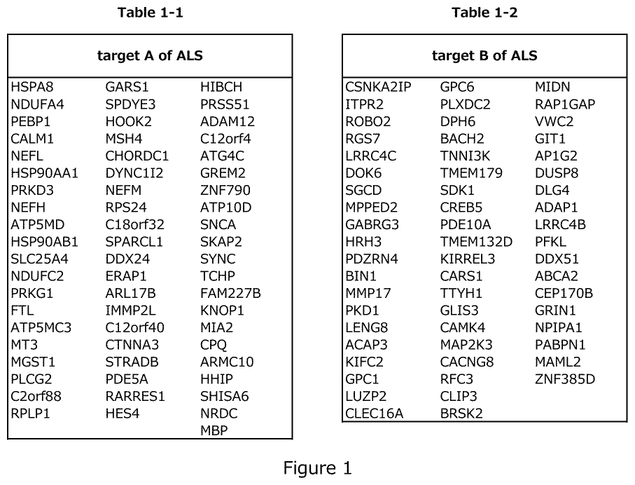

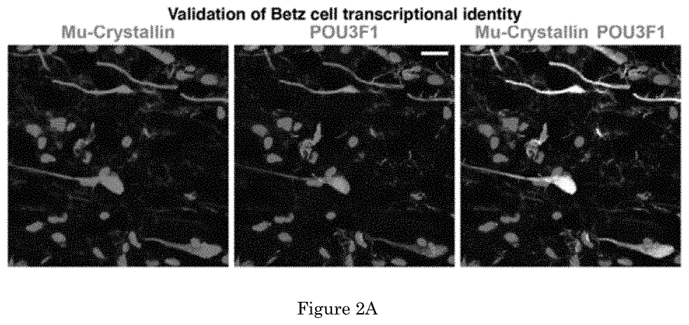

As described above, the ALS treatment method of the present invention includes administering to an ALS patient an inhibitor for the target A or a promoter for the target B. The target A is at least one gene selected from a group consisting of the genes in Table 1-1 or a protein encoded by the gene, and the inhibitor for the target A is a substance that inhibits expression of the gene, or a substance that inhibits a function of the protein encoded by the gene. The target B is at least one gene selected from a group consisting of the genes in Table 1-2 or a protein encoded by the gene, and the promoter for the target B is a substance that promotes the expression of the gene, or a substance that promotes a function of the protein encoded by the gene. FIG. 1 shows a total of 119 types of genes in Table 1-1 and Table 1-2, and the genes are numbered (No. 1-No. 119, see Table 7).

As described above, as a result of intensive studies, the inventors of the present invention have found that there are targets with high expression and targets with low expression in ALS patients as compared to a non-ALS patient such as healthy subject and ALS can be treated by inhibiting the former and promoting the latter. The present invention is characterized in that an inhibitor for the target A or a promoter for the target B is used for ALS patients, and other constitutions and conditions are not particularly limited.

In the present invention, for example, the ALS pharmaceutical composition of the present invention to be described later can be used as the inhibitor for the target A and/or the promoter for the target B (hereinafter, also referred to as the drug of the present invention). Unless otherwise specified, the ALS treatment method of the present invention can incorporate, for example, the description of the ALS pharmaceutical composition and other categories of the present invention to be described later.

In the ALS treatment method of the present invention, in the administration, as the drug, the inhibitor for the target A may be administered, the promoter for the target B may be administered, or both may be administered. In this way, in the administration, either the inhibitor or the promoter may be administered. However, for example, information about the expression level of the target A or the expression level of the target B in a biological sample of a patient to be administered and treated can be acquired and determination can be performed based on the information.

That is, when the expression level of the target A is acquired as the information of the biological sample of the patient to be administered, as illustrated in (A1) or (A2), the administration of the inhibitor for the target A can be determined based on an evaluation criterion.

(A1) When an expression level of the target A in a healthy subject is used as an evaluation criterion C1 and the expression level of the target A in the patient to be administered is higher than the evaluation criterion C1, the inhibitor for the target A is administered to the patient.

(A2) When an expression level of the target A in an ALS patient is used as an evaluation criterion C2, and the expression level of the target A in the patient is not significantly different or is high with respect to the evaluation criterion C2, the inhibitor for the target A is administered.

On the other hand, when the expression level of the target B is acquired as the information of the biological sample of the patient, as illustrated in (B1) or (B2), the administration of the promoter for the target B can be determined based on an evaluation criterion.

(B1) When an expression level C3 of the target B in a healthy subject is used as an evaluation criterion and the expression level of the target B in the patient is lower than the evaluation criterion C3, the promoter for the target B is administered to the patient.

(B2) When an expression level of the target B in an ALS patient is used as an evaluation criterion C4, and the expression level of the target B in the patient is not significantly different or is lower with respect to the evaluation criterion C4, the promoter for the target B is administered.

Further, it is also possible that, as the information of the biological sample of the patient, information about both the target A and the target B is acquired, and, from evaluation of at least one of (A1) and (A2) and evaluation of at least one of (B1) and (B2), administration of the inhibitor for the target A and the promoter for the target B is determined.

In the determination of the administration, the expression level of the target A may be, for example, an expression level of any one of multiple targets to be described later, or an expression level of two or more of the targets. Further, similarly, the expression level of the target B may also be, for example, an expression level of any one of multiple targets to be described later, or an expression level of two or more of the targets. In the present specification, that an expression level is higher than an evaluation criterion or that an expression level is lower than an evaluation criterion preferably means that it is significantly higher or it is significantly lower (hereinafter, the same applies). The degree of significance can be appropriately set. For example, when making a stricter determination, a relatively large significant difference can be set, and when making a looser determination, a relatively small significant difference can be set.

For the expression level of the healthy subject and the expression level of the ALS patient, which are the evaluation criteria, for example, information acquired in advance from the healthy subject and the ALS patient can be used. Further, the expression level of the patient to be administered and the expression levels of the healthy subject and the ALS patient that are the evaluation criteria are preferably, for example, expression levels of targets of the same type obtained from biological samples of the same type. The biological samples are each, for example, a sample derived from the brain, specifically, a cerebrospinal fluid.

As will be described later, the target A and the target B may each be a gene or a protein encoded by the gene. Therefore, the expression level may be, for example, an expression level of the gene or an expression level of the protein.

A method for measuring the expression level is not particularly limited, and, for example, a commonly known method used for analysis of gene expression and protein expression can be used. For the expression of the gene, for example, quantitative PCR, microarray, RNA sequence, and the like can be used, and for the expression of the protein, for example, ELISA, Western blotting, mass spectrometry, and the like can be used.

Next, the target A and its inhibitor, the target B and its promoter are illustrated below.

The target A is, for example, a target having a high expression level in an ALS patient as compared to a non-ALS patient. The target A is at least one gene selected from a group consisting of the genes in Table 1-1 below (hereinafter, also referred to as a gene A), or a protein encoded by the gene (hereinafter, also referred to as a protein A). That is, the target A in the present invention may be a gene shown in Table 1-1 below, or may be a protein encoded by the gene.

TABLE 1-1
target A of ALS

| | | |
|---|---|---|
| HSPA8 | GARS1 | HIBCH |
| NDUFA4 | SPDYE3 | PRSS51 |
| PEBP1 | HOOK2 | ADAM12 |
| CALM1 | MSH4 | C12orf4 |
| NEFL | CHORDC1 | ATG4C |
| HSP90AA1 | DYNC1I2 | GREM2 |
| PRKD3 | NEFM | ZNF790 |
| NEFH | RPS24 | ATP10D |
| ATP5MD | C18orf32 | SNCA |
| HSP90AB1 | SPARCL1 | SKAP2 |
| SLC25A4 | DDX24 | SYNC |
| NDUFC2 | ERAP1 | TCHP |
| PRKG1 | ARL17B | FAM227B |
| FTL | IMMP2L | KNOP1 |
| ATP5MC3 | C12orf40 | MIA2 |
| MT3 | CTNNA3 | CPQ |
| MGST1 | STRADB | ARMC10 |
| PLCG2 | PDE5A | HHIP |
| C2orf88 | RARRES1 | SHISA6 |
| RPLP1 | HES4 | NRDC |
| | | MBP |

The inhibitor for the target A may be, for example, a substance that inhibits expression of the gene (hereinafter, also referred to as an expression inhibitor), or may be a substance that inhibits a function of the protein (hereinafter, also referred to as a function inhibitor), or both may be used in combination. As described above, the present invention is characterized in that the target A showing high expression in ALS patients is found, and therefore, there are no restrictions on the type of the inhibitor for the target A, an inhibitory method, and the like.

The expression inhibitor may be, for example, a substance that inhibits transcription or a substance that inhibits translation in expression of the protein A from the gene A. Examples of transcription inhibition include inhibition of transcription from DNA to a mRNA precursor (pre-mRNA), inhibition of RNA processing (for example, splicing) in which a mature mRNA is formed from a mRNA precursor, degradation of a mRNA precursor or a mature mRNA, and the like. Examples of translational inhibition include inhibition of translation from a mature mRNA, inhibition of modification of a translation product, and the like.

An example of the expression inhibitor is a nucleic acid substance (hereinafter, also referred to as a nucleic acid-type expression inhibitor). The expression inhibitor may be, for example, in a first form in which expression is inhibited by the inhibitor as it is, or in a second form of a precursor, which is in a state in which expression is inhibited in an in vivo or in vitro environment.

Examples of the expression inhibitor of the first form include an interfering nucleic acid (for example, an RNAi substance), an antisense (antisense oligonucleotide), an antigene, a ribozyme, and the like. Examples of the RNAi substance include siRNA, miRNA, and the like. The antisense and the miRNA, for example, inhibit translation from mRNA, the siRNA and the ribozyme, for example, degrade mRNA, and the antigene, for example, inhibits transcription of mRNA. For these expression inhibitors, for example, either an entire region or a partial region of a target gene may be a target region. As specific examples, the antisense and the miRNA can be designed, for example, to bind to a 3′UTR region of mRNA transcribed from a target gene, and the siRNA and the ribozyme can be designed, for example, to bind completely complementaryly to a partial region of mRNA transcribed from a target gene.

The expression inhibitor may be, for example, a single-stranded oligonucleotide or a double-stranded oligonucleotide. A structural unit of the expression inhibitor is not particularly limited, and is, for example, a nucleotide residue or a non-nucleotide residue. Examples of the former include a deoxyribonucleotide skeleton or a ribonucleotide skeleton that contain a sugar, a base such as purine or pyrimidine, and a phosphoric acid, and examples of the latter include a non-nucleotide skeleton that contains a base such as pyrrolidine or piperidine. The structural unit may be, for example, of a natural type or an artificial non-natural type. The expression inhibitor may be formed from, for example, the same structural unit, or may be formed from two or more types of structural units. The expression inhibitor may be, for example, of a modified type or an unmodified type, and in the former case, for example, any of a sugar, a base, an internucleoside linkage and the like may be modified.

Examples of the expression inhibitor of the second form include a precursor that expresses an expression inhibitor of the first form in an in vivo or in vitro environment. When administered to a subject, the precursor, for example, can express an expression inhibitor of the first form and exert an expression-inhibiting function.

An example of the precursor is a form that contains an expression inhibitor of the first form and a linker. A specific example of the precursor is a form in which both strands of siRNA are linked by the linker. According to such a precursor, for example, by cleaving the precursor in an in vivo, in vitro, or ex vivo environment, the linker can be removed from the precursor and a double-stranded siRNA can be produced (expressed). As a specific example of the precursor, for example, shRNA or the like that produces siRNA by cleavage can be exemplified.

Further, the precursor may be, for example, an expression vector into which a coding sequence of an expression inhibitor of the first form is inserted. According to the expression vector, for example, an expression inhibitor of the first form can be expressed in an in vivo or in vitro environment. A type of the expression vector is not particularly limited, and examples thereof include a plasmid vector, a viral vector, and the like, and examples of the viral vector include an adenovirus vector, a Sendai viral vector, and the like.

Examples of the function inhibitor include an activity inhibitor that inhibits activity of the protein and an activity neutralizer that neutralizes the activity of the protein. The function inhibitor may be, for example, in a first form in which activity is inhibited by the inhibitor as it is, or in a second form of a precursor, which is in a state in which activity is inhibited in an in vivo or in vitro environment.

The activity inhibitor is not particularly limited, and examples thereof include a low molecular weight compound and the like.

Examples of the activity neutralizer of the first form include an antibody or an antigen-binding fragment (antigen-binding peptide) against the protein, and the like. These can inhibit the function of the protein, for example, by binding to the protein, and thus are also referred to a neutralizing antibody or a neutralizing antigen-binding fragment. The antibody may be, for example, a monoclonal antibody or a polyclonal antibody, and its isotype is not particularly limited, and examples of the isotype include IgG, IgM, IgA, and the like. When the antibody is administered to a human, for example, a fully human antibody, a humanized antibody, a chimeric antibody and the like are preferable. The antigen-binding fragment, for example, may be able to recognize and bind to a target site of the target protein, and an example thereof is a fragment having a complementarity-determining region (CDR) of the antibody. Specific examples of the antigen-binding fragment include fragments such as Fab, Fab′, and F (ab′), and the like.

In addition, the examples of the activity neutralizer also include an aptamer (nucleic acid aptamer) for the protein, and the like. Similar to the antibody and the like, the aptamer can inhibit the function of the protein by binding to the protein, and thus is also referred to as a neutralization aptamer. A structural unit of the aptamer is, for example, a deoxyribonucleotide skeleton, a ribonucleotide skeleton, a non-nucleotide skeleton, or the like, and the description of the expression inhibitor of the first form can be incorporated.

An example of the activity neutralizer of the second form is a precursor that expresses an activity neutralizer of the first form in an in vivo or in vitro environment. When administered to a subject, the precursor, for example, can express an activity neutralizer of the first form and exert an activity-inhibiting function.

The precursor may be, for example, an expression vector into which a coding sequence of the antibody or the antigen-binding fragment is inserted. According to the expression vector, for example, the antibody or the antigen-binding fragment can be expressed in an in vivo or in vitro environment. A type of the expression vector is not particularly limited, and examples thereof include a plasmid vector, a viral vector, and the like, and examples of the viral vector include an adenovirus vector, an adeno-associated virus vector, a lentiviral vector, a Sendai viral vector, and the like.

The target B is, for example, a target having a low expression level in an ALS patient as compared to a non-ALS patient. The target B is at least one gene selected from a group consisting of the genes in Table 1-2 below (hereinafter, also referred to as a gene B), or a protein encoded by the gene (hereinafter, also referred to as a protein B). That is, the target B in the present invention may be a gene shown in Table 1-2 below, or may be a protein encoded by the gene.

TABLE 1-2
target B of ALS

| | | |
|---|---|---|
| CSNKA2IP | GPC6 | MIDN |
| ITPR2 | PLXDC2 | RAP1GAP |
| ROBO2 | DPH6 | VWC2 |
| RGS7 | BACH2 | GIT1 |
| LRRC4C | TNNI3K | AP1G2 |
| DOK6 | TMEM179 | DUSP8 |
| SGCD | SDK1 | DLG4 |
| MPPED2 | CREB5 | ADAP1 |
| GABRG3 | PDE10A | LRRC4B |
| HRH3 | TMEM132D | PFKL |
| PDZRN4 | KIRREL3 | DDX51 |
| BIN1 | CARS1 | ABCA2 |
| MMP17 | TTYH1 | CEP170B |
| PKD1 | GLIS3 | GRIN1 |
| LENG8 | CAMK4 | NPIPA1 |
| ACAP3 | MAP2K3 | PABPN1 |
| KIFC2 | CACNG8 | MAML2 |
| GPC1 | RFC3 | ZNF385D |
| LUZP2 | CLIP3 | |
| CLEC16A | BRSK2 | |

The promoter for the target B may be, for example, a substance that promotes expression of the gene (hereinafter, also referred to as an expression promoter), or may be a substance that promotes a function of the protein (hereinafter, also referred to as a function promoter), or both may be used in combination. As described above, the present invention is characterized in that the target B showing low expression in ALS patients is found, and therefore, there are no restrictions on the type of the promoter for the target B, an promoting method, and the like.

The expression promoter may be, for example, a substance that promotes either transcription or translation in expression of the protein B from the gene B. Further, in the present invention, the promotion of expression may be, for example, promotion of transcription of the gene B inherent in a living body or promotion of production of the protein B by translation, and may be promotion by administering the gene B or the protein B to a living body.

The expression promoter is, for example, a vector that expresses the gene B, and a specific example thereof is an expression vector into which a coding sequence of the gene B is inserted. According to the expression vector, for example, in an in vivo or in vitro environment, the gene B can be transcribed by the expression vector, and further, a protein can be produced by translation. A type of the expression vector is not particularly limited, and examples thereof include a plasmid vector, a viral vector, and the like, and examples of the viral vector include an adenovirus vector, an adeno-associated virus vector, a lentiviral vector, a Sendai viral vector, and the like.

In the ALS treatment method of the present invention, for example, based on genetic information of a patient to be administered and treated, it is possible to further administer the more suitable drug (the inhibitor or the promoter).

(1) When a Patient has No ALS-Related Mutation for a Specific Gene

It is known that some ALS patients are classified into a group that shows specific mutations for a specific gene cluster (a C9orf72 gene, a SOD1 gene, a TBK1 gene, a TARDBP gene, a FUS gene and a NEK1 gene). As a result of further studies, the present inventors have found that when an ALS patient does not have the specific mutation for the specific gene cluster, among the targets A, expression of the following target A1 is high, and among the targets B, expression of the following target B1 is low. Therefore, when a patient to be administered does not have the mutation, for example, a more effective therapeutic effect can be obtained by administering at least one of an inhibitor for the target A1 and a promoter for the target B1.

The target A1 is, for example, at least one gene selected from a group consisting of the genes in Table 2-1 below (hereinafter, also referred to as a gene A1), or a protein encoded by the gene (hereinafter, also referred to as a protein A1). Further, the target B1 is, for example, at least one gene selected from a group consisting of the genes in Table 2-2 below (hereinafter, also referred to as a gene B1), or a protein encoded by the gene (hereinafter, also referred to as a protein B1).

TABLE 2-1
target A1 of ALS
(C9orf72_wt)

| | | |
|---|---|---|
| HSPA8 | HOOK2 | ARMC10 |
| NDUFA4 | DYNC1I2 | HHIP |
| PEBP1 | NEFM | SHISA6 |
| CALM1 | RPS24 | NRDC |
| NEFL | C18orf32 | |
| HSP90AA1 | SPARCL1 | |
| PRKD3 | DDX24 | |
| NEFH | ERAP1 | |
| ATP5MD | ARL17B | |
| HSP90AB1 | C12orf40 | |
| SLC25A4 | CTNNA3 | |
| NDUFC2 | STRADB | |
| PRKG1 | PDE5A | |
| ATP5MC3 | ZNF790 | |
| MGST1 | ATP10D | |
| PLCG2 | SYNC | |
| C2orf88 | FAM227B | |
| RPLP1 | KNOP1 | |
| GARS1 | MIA2 | |
| SPDYE3 | CPQ | |

TABLE 2-2
target B1 of ALS
(C9orf72_wt)

| | | |
|---|---|---|
| ITPR2 | MAP2K3 | MAML2 |
| LRRC4C | CACNG8 | ZNF385D |
| DOK6 | RFC3 | |
| HRH3 | CLIP3 | |
| PDZRN4 | BRSK2 | |
| BIN1 | MIDN | |
| MMP17 | RAP1GAP | |
| PKD1 | GIT1 | |
| LENG8 | AP1G2 | |
| ACAP3 | DUSP8 | |
| KIFC2 | DLG4 | |
| GPC1 | ADAP1 | |
| LUZP2 | LRRC4B | |
| BACH2 | PFKL | |
| TNNI3K | DDX51 | |
| TMEM179 | ABCA2 | |
| SDK1 | CEP170B | |
| CREB5 | GRIN1 | |
| TMEM132D | NPIPA1 | |
| KIRREL3 | PABPN1 | |

Mutations of the gene cluster include, for example, abnormal elongation of a 6-base repeated sequence in intron 1 in the C9orf72 gene, A4V in the SOD1 gene, T4A in the TBK1 gene, A382T in the TARDBP gene, P525L in the FUS gene, and R261H in the NEK1 gene.

(2) When a Patient has an ALS-Related Mutation for a Specific Gene

As a result of further studies, the present inventors have found that when an ALS patient has the specific mutation for the specific gene cluster, among the targets A, expression of the following target A2 is high, and among the targets B, expression of the following target B2 is low. Therefore, when a patient to be administered has the mutation, for example, a more effective therapeutic effect can be obtained by administering at least one of an inhibitor for the target A2 and a promoter for the target B2.

The target A2 is, for example, at least one gene selected from a group consisting of the genes in Table 3-1 below (hereinafter, also referred to as a gene A2), or a protein encoded by the gene (hereinafter, also referred to as a protein A2). Further, the target B2 is, for example, at least one gene selected from a group consisting of the genes in Table 3-2 below (hereinafter, also referred to as a gene B2), or a protein encoded by the gene (hereinafter, also referred to as a protein B2).

TABLE 3-1
target A2 of ALS
(C9orf72_mt)

| | | |
|---|---|---|
| NDUFA4 | DDX24 | KNOP1 |
| CALM1 | IMMP2L | ARMC10 |
| NEFL | C12orf40 | SHISA6 |
| PRKD3 | CTNNA3 | NRDC |
| ATP5MD | STRADB | MBP |
| SLC25A4 | RARRES1 | |
| FTL | HES4 | |
| MT3 | HIBCH | |
| MGST1 | PRSS51 | |
| PLCG2 | ADAM12 | |
| C2orf88 | C12orf4 | |
| RPLP1 | ATG4C | |
| GARS1 | GREM2 | |
| SPDYE3 | ZNF790 | |
| HOOK2 | ATP10D | |
| MSH4 | SNCA | |
| CHORDC1 | SKAP2 | |
| DYNC1I2 | SYNC | |
| NEFM | TCHP | |
| RPS24 | FAM227B | |

TABLE 3-2
target B2 of ALS
(C9orf72_mt)

| | | |
|---|---|---|
| CSNKA2IP | SDK1 | PFKL |
| ROBO2 | CREB5 | ABCA2 |
| RGS7 | PDE10A | CEP170B |
| DOK6 | TMEM132D | GRIN1 |
| SGCD | KIRREL3 | NPIPA1 |
| MPPED2 | CARS1 | PABPN1 |
| GABRG3 | TTYH1 | MAML2 |
| PDZRN4 | GLIS3 | ZNF385D |
| BIN1 | CAMK4 | |
| MMP17 | MAP2K3 | |
| PKD1 | CACNG8 | |
| KIFC2 | RFC3 | |
| GPC1 | CLIP3 | |
| LUZP2 | BRSK2 | |
| CLEC16A | MIDN | |
| GPC6 | RAP1GAP | |
| PLXDC2 | VWC2 | |
| DPH6 | DUSP8 | |
| BACH2 | DLG4 | |
| TMEM179 | LRRC4B | |

(3) When it is Unknown Whether or not a Patient has an ALS-Related Mutation for a Specific Gene

As a result of further studies, the present inventors have found that whether an ALS patient has the specific mutation or does not have the specific mutation for the specific gene cluster, among the targets A, expression of the following target A3 is high, and among the targets B, expression of the following target B3 is low. Therefore, when it is unknown whether or not a patient to be administered has the mutation, for example, a more effective therapeutic effect can be obtained by administering at least one of an inhibitor for the target A3 and a promoter for the target B3. Further, even when whether or not the patient has the mutation is known, since an effective treatment is possible regardless of whether the patient has the mutation, determination in treatment can be more easily performed.

The target A3 is, for example, at least one gene selected from a group consisting of the genes in Table 4-1 below (hereinafter, also referred to as a gene A3), or a protein encoded by the gene (hereinafter, also referred to as a protein A3). Further, the target B3 is, for example, at least one gene selected from a group consisting of the genes in Table 4-2 below (hereinafter, also referred to as a gene B3), or a protein encoded by the gene (hereinafter, also referred to as a protein B3).

TABLE 4-1
target A3 of ALS
(C9orf72_wt & C9orf72_mt)

| | |
|---|---|
| NDUFA4 | ZNF790 |
| CALM1 | ATP10D |
| NEFL | SYNC |
| PRKD3 | FAM227B |
| ATP5MD | KNOP1 |
| SLC25A4 | ARMC10 |
| MGST1 | SHISA6 |
| PLCG2 | NRDC |
| C2orf88 | |
| RPLP1 | |
| GARS1 | |
| SPDYE3 | |
| HOOK2 | |
| DYNC1I2 | |
| NEFM | |
| RPS24 | |
| DDX24 | |
| C12orf40 | |
| CTNNA3 | |
| STRADB | |

TABLE 4-2
target B3 of ALS
(C9orf72_wt & C9orf72_mt)

| | |
|---|---|
| DOK6 | RAP1GAP |
| PDZRN4 | DUSP8 |
| BIN1 | DLG4 |
| MMP17 | LRRC4B |
| PKD1 | PFKL |
| KIFC2 | ABCA2 |
| GPC1 | CEP170B |
| LUZP2 | GRIN1 |
| BACH2 | NPIPA1 |
| TMEM179 | PABPN1 |
| SDK1 | MAML2 |
| CREB5 | ZNF385D |
| TMEM132D | |
| KIRREL3 | |
| MAP2K3 | |
| CACNG8 | |
| RFC3 | |
| CLIP3 | |
| BRSK2 | |
| MIDN | |

The genes in Tables 1 to 4 described above and in Tables 5 and 6 to be described below are listed in Table 7 (7-1, 7-2) below. Table 7 shows numbers and names of the 119 genes, which are marked with circles to indicate which of Tables 1 to 6 they are in.

TABLE 7-1

| | | | | | Table4-1 | Table5-1 | Table6-1 |
|---|---|---|---|---|---|---|---|
| | | | Table2-1 | Table3-1 | target A3 | target A4 | target A5 |
| | | Table1-1 | target A1 | target A2 | of ALS | of ALS | of ALS |
| | | target A | of ALS | of ALS | (C9orf72_wt & | (only | (only |
| No. | Name | of ALS | (C9orf72_wt) | (C9orf72_mt) | C9orf72_mt) | C9orf72_wt) | C9orf72_mt) |
| 1 | HSPAB | ◯ | ◯ | | | ◯ | |
| 2 | NDUFA4 | ◯ | ◯ | ◯ | ◯ | | |
| 3 | PEBP1 | ◯ | ◯ | | | ◯ | |
| 4 | CALM1 | ◯ | ◯ | ◯ | ◯ | | |
| 5 | NEFL | ◯ | ◯ | ◯ | ◯ | | |
| 6 | HSP90AA1 | ◯ | ◯ | | | ◯ | |
| 7 | PRKD3 | ◯ | ◯ | ◯ | ◯ | | |
| 8 | NEFH | ◯ | ◯ | | | ◯ | |
| 9 | ATP5MD | ◯ | ◯ | ◯ | ◯ | | |
| 10 | HP90AB1 | ◯ | ◯ | | | ◯ | |
| 11 | SLC25A4 | ◯ | ◯ | ◯ | ◯ | | |
| 12 | NDUFC2 | ◯ | ◯ | | | ◯ | |
| 13 | PRKG1 | ◯ | ◯ | | | ◯ | |
| 14 | FTL | ◯ | | ◯ | | | ◯ |
| 15 | ATP5MC3 | ◯ | ◯ | | | ◯ | |
| 16 | MT3 | ◯ | | ◯ | | | ◯ |
| 17 | MGST1 | ◯ | ◯ | ◯ | ◯ | | |
| 18 | PLOG2 | ◯ | ◯ | ◯ | ◯ | | |
| 19 | C2orf88 | ◯ | ◯ | ◯ | ◯ | | |
| 20 | RPLP3 | ◯ | ◯ | ◯ | ◯ | | |
| 21 | GARS1 | ◯ | ◯ | ◯ | ◯ | | |
| 22 | 5PDYE3 | ◯ | ◯ | ◯ | ◯ | | |
| 23 | HOOK2 | ◯ | ◯ | ◯ | ◯ | | |
| 24 | MSH4 | ◯ | | ◯ | | | ◯ |
| 25 | CHORDC1 | ◯ | | ◯ | | | ◯ |
| 26 | DYNC1I2 | ◯ | ◯ | ◯ | ◯ | | |
| 27 | NEFM | ◯ | ◯ | ◯ | ◯ | | |
| 28 | RP524 | ◯ | ◯ | ◯ | ◯ | | |
| 29 | C18orf32 | ◯ | ◯ | | | ◯ | |
| 30 | SPARCL1 | ◯ | ◯ | | | ◯ | |
| 31 | DOX24 | ◯ | ◯ | ◯ | ◯ | | |
| 32 | ERAP1 | ◯ | ◯ | | | ◯ | |
| 33 | ARL178 | ◯ | ◯ | | | ◯ | |
| 35 | IMMP2L | ◯ | | ◯ | | | ◯ |
| 66 | C12orf40 | ◯ | ◯ | ◯ | ◯ | | |
| 67 | CTNNA3 | ◯ | ◯ | ◯ | ◯ | | |
| 68 | STRAD8 | ◯ | ◯ | ◯ | ◯ | | |
| 69 | POE5A | ◯ | ◯ | | | ◯ | |
| 70 | RARRES1 | ◯ | | ◯ | | | |
| 71 | HES4 | ◯ | | ◯ | | | ◯ |
| 72 | HIBCH | ◯ | | ◯ | | | ◯ |
| 73 | PRSSS1 | ◯ | | ◯ | | | |
| 74 | ADAM12 | ◯ | | ◯ | | | ◯ |
| 75 | C12orf4 | ◯ | | ◯ | | | ◯ |
| 76 | ATG4C | ◯ | | ◯ | | | ◯ |
| 77 | GREM2 | ◯ | | ◯ | | | ◯ |
| 78 | ZNF790 | ◯ | ◯ | ◯ | ◯ | | |
| 79 | ATP10D | ◯ | ◯ | ◯ | ◯ | | |
| 80 | SNCA | ◯ | | ◯ | | | ◯ |
| 81 | SKAP2 | ◯ | | ◯ | | | ◯ |
| 82 | SYNC | ◯ | ◯ | ◯ | ◯ | | |
| 83 | TCHP | ◯ | | ◯ | | | ◯ |
| 84 | FAM2278 | ◯ | ◯ | ◯ | ◯ | | |
| 85 | KNOP1 | ◯ | ◯ | ◯ | ◯ | | |
| 86 | MIA2 | ◯ | ◯ | | | ◯ | |
| 87 | CPQ | ◯ | ◯ | | | ◯ | |
| 88 | ARMC10 | ◯ | ◯ | ◯ | ◯ | | |
| 89 | HHIP | ◯ | ◯ | | | ◯ | |
| 90 | SKISA6 | ◯ | ◯ | ◯ | ◯ | | |
| 91 | NRDC | ◯ | ◯ | ◯ | ◯ | | |
| 92 | MBP | ◯ | | ◯ | | | ◯ |

TABLE 7-2

| | | | | | Table4-2 | Table5-2 | Table6-2 |
|---|---|---|---|---|---|---|---|
| | | | Table2-2 | Table3-2 | target B3 | target B4 | target B5 |
| | | Table1-2 | target B1 | target B2 | of ALS | of ALS | of ALS |
| | | target B | of ALS | of ALS | (C9orf72_wt & | (only | (only |
| No. | Name | of ALS | (C9orf72_wt) | (C9orf72_mt) | CPorf72_mt) | C9orf72_wt) | C9orf72_mt) |
| 34 | CSNKA2IP | ◯ | | ◯ | | | ◯ |
| 36 | ITPR2 | ◯ | ◯ | | | ◯ | |
| 37 | ROBO2 | ◯ | | ◯ | | | ◯ |
| 38 | RGS7 | ◯ | | ◯ | | | ◯ |
| 39 | LRRC4C | ◯ | ◯ | | | ◯ | |
| 40 | DOK6 | ◯ | ◯ | ◯ | ◯ | | |
| 41 | SGCD | ◯ | | ◯ | | | ◯ |
| 42 | MPPED2 | ◯ | | ◯ | | | ◯ |
| 43 | GABRG3 | ◯ | | ◯ | | | ◯ |
| 44 | HRH3 | ◯ | ◯ | | | ◯ | |
| 45 | PDZRN4 | ◯ | ◯ | ◯ | ◯ | | |
| 46 | B1N1 | ◯ | ◯ | ◯ | ◯ | | |
| 47 | MMP17 | ◯ | ◯ | ◯ | ◯ | | |
| 48 | PKD1 | ◯ | ◯ | ◯ | ◯ | | |
| 49 | LENG8 | ◯ | ◯ | | | ◯ | |
| 50 | ACAP3 | ◯ | ◯ | | | ◯ | |
| 51 | KIFC2 | ◯ | ◯ | ◯ | ◯ | | |
| 52 | GPC1 | ◯ | ◯ | ◯ | ◯ | | |
| 53 | LUZP2 | ◯ | ◯ | ◯ | ◯ | | |
| 54 | CLEC16A | ◯ | | ◯ | | | ◯ |
| 55 | GPC6 | ◯ | | ◯ | | | ◯ |
| 56 | PLXDC2 | ◯ | | ◯ | | | ◯ |
| 57 | DPH6 | ◯ | | ◯ | | | ◯ |
| 58 | BACH2 | ◯ | ◯ | ◯ | ◯ | | |
| 59 | TNNI3K | ◯ | ◯ | | | ◯ | |
| 60 | TMEM179 | ◯ | ◯ | ◯ | ◯ | | |
| 61 | SDK1 | ◯ | ◯ | ◯ | ◯ | | |
| 62 | CREB5 | ◯ | ◯ | ◯ | ◯ | | |
| 63 | PDE10A | ◯ | | ◯ | | | ◯ |
| 64 | TMEM132D | ◯ | ◯ | ◯ | ◯ | | |
| 65 | KIRREL3 | ◯ | ◯ | ◯ | ◯ | | |
| 93 | CARS1 | ◯ | | ◯ | | | ◯ |
| 94 | TTYH1 | ◯ | | ◯ | | | ◯ |
| 95 | GLIS3 | ◯ | | ◯ | | | ◯ |
| 96 | CAMK4 | ◯ | | ◯ | | | ◯ |
| 97 | MAP2K3 | ◯ | ◯ | ◯ | ◯ | | |
| 98 | CACNG8 | ◯ | ◯ | ◯ | ◯ | | |
| 99 | RFC3 | ◯ | ◯ | ◯ | ◯ | | |
| 100 | CLIP3 | ◯ | ◯ | ◯ | ◯ | | |
| 101 | BRSK2 | ◯ | ◯ | ◯ | ◯ | | |
| 102 | MIDN | ◯ | ◯ | ◯ | ◯ | | |
| 103 | RAP1GAP | ◯ | ◯ | ◯ | ◯ | | |
| 104 | VWC2 | ◯ | | ◯ | | | ◯ |
| 105 | GIT1 | ◯ | ◯ | | | ◯ | |
| 106 | AP1G2 | ◯ | ◯ | | | ◯ | |
| 107 | DUSP8 | ◯ | ◯ | ◯ | ◯ | | |
| 108 | DLG4 | ◯ | ◯ | ◯ | ◯ | | |
| 109 | ADAP1 | ◯ | ◯ | | | ◯ | |
| 110 | LRRC4B | ◯ | ◯ | ◯ | ◯ | | |
| 111 | PFKL | ◯ | ◯ | ◯ | ◯ | | |
| 112 | DDX51 | ◯ | ◯ | | | ◯ | |
| 113 | ABCA2 | ◯ | ◯ | ◯ | ◯ | | |
| 114 | CEP170B | ◯ | ◯ | ◯ | ◯ | | |
| 115 | GRIN1 | ◯ | ◯ | ◯ | ◯ | | |
| 116 | NPIPA1 | ◯ | ◯ | ◯ | ◯ | | |
| 117 | PABPN1 | ◯ | ◯ | ◯ | ◯ | | |
| 118 | MAML2 | ◯ | ◯ | ◯ | ◯ | | |
| 119 | ZNF38SD | ◯ | ◯ | ◯ | ◯ | | |

In the ALS treatment method of the present invention, in the administration, a method for administering the drug (the inhibitor for the target A or the promoter for the target B) is not particularly limited.

Examples of a patient to be administered include a patient diagnosed with ALS, a patient with ALS symptoms, a patient with suspected ALS, and the like. Regarding the patient, for example, preferably, prior to the administration, information about an expression level of at least one of the target A and the target B in a biological sample collected from the patient is acquired, and, as described above, the drug to be administered is further selected based on various evaluation criteria. Further, regarding the patient, for example, preferably, prior to administration, information about an ALS-specific mutation in the specific gene is acquired, and, as described above, the drug to be administered is further selected based on presence or absence of a mutation or whether or not presence or absence of a mutation is unknown.

In the administration, the method for administering the drug is not particularly limited, for example, as long as the drug is finally delivered to a target affected area. Specifically, for example, it is possible that the affected area is set as an administration area and the drug is directly administered to the affected area, or an area different from the affected area is set as an administration area, and the drug is indirectly administered so as to be delivered from the administration area to the affected area. The affected area is, for example, the brain, specifically, the motor cortex.

The method for administering the drug is not particularly limited, and examples thereof include parenteral administration, oral administration, and the like. Examples of the parenteral administration method include affected area administration, intravenous administration, subcutaneous administration, intradermal administration, intramuscular administration, nasal administration, transdermal administration, intrathecal administration, gastric fistula administration, and the like. Since ALS is a disease that generally develops due to abnormalities in the nerves of the brain, a directly affected area of treatment is, for example, preferably the brain as described above. As methods for administering the drug in which the brain is an affected area, for example, direct administration to the brain, nasal administration, intrathecal administration, and the like are preferable.

The administration step may include administering the composition containing only at least one of the active ingredients, or administering the composition containing at least one of the active ingredients and other additives. The active ingredients (the inhibitor for the target A or the promoter for the target B) may include only the drug, or the drug and other active ingredients. The composition for ALS treatment in this invention as described later is similar. A dosage form of the drug is not particularly limited, and examples thereof include liquid, cream, gel, powder, solid, and the like. Specific examples of the drug in the case of parenteral administration include infusion, injection, transdermal preparation, transmucosal preparation, nasal spray, inhalant, suppository, and the like. Further, examples of the drug in the case of oral administration include solutions, suspensions, emulsions, syrups, pills, granules, fine granules, powders, capsules (hard capsules, soft capsules), and the like. When the form of the drug at the time of administration is a liquid such as the infusion or the injection, the form of the drug before administration is not limited to this, and may be, for example, a concentrate, a powder prepared by freeze-drying or the like, a granule liquid, or the like. In this case, the liquid drug may be prepared by diluting, dissolving or suspending the drug in an aqueous medium such as physiological saline at the time of use. As the drug, as described above, for example, the pharmaceutical composition of the present invention can be used, and, as specific examples of its composition and the like, the description of the pharmaceutical composition of the present invention to be described later can be incorporated.

In the ALS treatment method of the present invention, conditions for administering the drug are not particularly limited and can be appropriately determined according to, for example, the type of disease of the patient, the severity (stage) of the disease, symptoms, age, gender, purpose of treatment (such as symptom relief, or prevention), and the like. Patients to be administered are, for example, humans or non-human animals, and examples of the non-human animals include mice, rats, monkeys, dogs and the like. In the treatment, for example, as the drug, a therapeutically effective amount of the active ingredient (at least one of the inhibitor and the promoter) is administered. As a specific example, when a patient is an adult weighing 60 kg, a daily dose of the active ingredient of the drug may be, for example, 0.1 ng to 100 mg, and the number of administrations per day may be, for example, once or twice or more. Further, the number of administrations during a treatment period may be, for example, continuously performed daily or intermittently performed every few days.

As described above, the present inventors have confirmed genes of which expression fluctuates in ALS patients as compared with healthy subjects. Specifically, as shown in Examples to be described later, for giant pyramidal tract upper motor neurons (UMN PT) known as Betz cells, gene expression analysis data was acquired and weighted gene correlational analysis (WGCNA) analysis was performed. As a result of this analysis, the genes could be classified into 40 types of gene modules. Then, a pathway analysis was performed on gene sets contained in each module, and it was found that three gene modules (Nos. 2, 5, and 11) contained significantly more genes contained in ALS-related pathways. Then, as shown in Table 8 below, these gene modules 2, 5 and 11 contained a total of 19 types of genes. Therefore, among the genes shown in Table 7 above, the 19 types of genes included in Table 8 below are presumed to be particularly useful as targets for the treatment of ALS. In Table 8, UP indicates a gene with high expression in ALS, and DOWN indicates a gene with low expression in ALS.

TABLE 8

| | | UP | DOWN |
|---|---|---|---|
| No. | Name | Module No. | Module No. |
| 1 | HSPA8 | 5 | 5 |
| 2 | NDUFA4 | 2 | 2 |
| 4 | CALM1 | 5 | 5 |
| 5 | NEFL | 5 | 5 |
| 8 | NEFH | 11 | |
| 9 | ATP5MD | 2 | |
| 11 | SLC25A4 | 5 | |
| 12 | NDUFC2 | 5 | |
| 15 | ATP5MC3 | 2 | |
| 27 | NEFM | 5 | |
| 29 | C18orf32 | 5 | |
| 30 | SPARCL1 | 5 | |
| 35 | IMMP2L | 2 | |
| 40 | DOK6 | | 2 |
| 43 | GABRG3 | | 5 |
| 57 | DPH6 | | 2 |
| 71 | HES4 | 2 | |
| 72 | HIBCH | 2 | |
| 118 | MAML2 | | 5 |

In the treatment method of the present invention, a target is not limited to the target A and the target B, and, for example, may be at least one gene selected from a group consisting of genes in Table 9 below (hereinafter, also referred to as a gene D) or a protein encoded by the gene (hereinafter also referred to as a protein D).

TABLE 9

| | | | | | |
|---|---|---|---|---|---|
| STMN1 | CHCHD10 | UBQLN2 | PRMT1 | VPS25 | ALKBH5 |
| STMN2 | SOD1 | PFN1 | CHMP4B | SRSF3 | CNOT7 |
| STMN3 | SQSTM1 | PRNP | CHMP1A | SRSF8 | RPP25 |
| STMN4 | VAPB | NUP50 | CHMP5 | EIF2AK2 | |
| DCTN1 | VCP | TNPO3 | VPS28 | RBM15B | |

As described above, ALS is a type of neurodegenerative disease, and other neurodegenerative diseases such as frontotemporal lobar degeneration (FTLD) are known. When a disease to be treated is FTLD, examples of a target include a gene in Example B to be described later and a protein encoded by the gene. Further, for example, when the expression level of the target is higher than that in a healthy subject, an inhibitor that inhibits the expression of the target can be used as an active ingredient, and when the expression level of the target is lower than that in a healthy subject, a promoter that promotes the expression of the target can be used as an active ingredient.

As described above, the treatment method of the present invention includes administering to an ALS patient a substance that promotes or inhibits expression of a gene involved in receptor diffusion trapping or a function of a protein encoded by the gene. Unless otherwise specified, the treatment method of the present invention can incorporate the above description.

As described above, as a result of intensive studies, the present inventors have found that there are targets with high expression and targets with low expression in ALS patients, and targets that exhibit such behaviors include genes involved in receptor diffusion trapping, or proteins encoded by the genes. Therefore, according to the present invention, ALS can be treated by promoting or inhibiting expression of a gene involved in receptor diffusion trapping, or a function of a protein encoded by the gene.

In the present invention, the receptor diffusion trapping is, for example, postsynaptic neurotransmitter receptor diffusion trapping or neurotransmitter receptor diffusion trapping. The genes involved in the receptor diffusion trapping are, for example, SHISA6, CACNG8, DLG4, and the like. SHISA6 is listed in Table 1-1, 2-1, 3-1, and 4-1 above, and its expression is high in ALS patients. Therefore, in the present invention, for example, it is preferable to administer a substance that inhibits expression of the gene or a function of a protein encoded by the gene. Further, CACNG8 and DLG4 are listed in Tables 1-2, 2-2, 3-2, and 4-2 above, and their expression is low in ALS patients. Therefore, in the present invention, for example, it is preferable to administer a substance that promotes the expression of the genes or functions of proteins encoded by the genes.

### ALS Therapeutic Composition

As described above, the ALS therapeutic composition of the present invention contains an inhibitor for a target A or a promoter for a target B. The target A is at least one gene selected from a group consisting of genes in Table 1-1 or a protein encoded by the gene.

The inhibitor for the target A is a substance that inhibits expression of the gene, or a substance that inhibits a function of the protein encoded by the gene.

The target B is at least one gene selected from a group consisting of genes in Table 1-2 or a protein encoded by the gene.

The promoter for the target B is a substance that promotes expression of the gene, or a substance that promotes a function of the protein encoded by the gene.

The ALS therapeutic composition of the present invention is characterized in that at least one of an inhibitor for the target A and a promoter for the target B is contained, and other constitutions and conditions are not particularly limited. Unless otherwise specified, the ALS therapeutic composition of the present invention can incorporate the description of the ALS treatment method of the present invention.

The ALS therapeutic composition of the present invention may contain, as an active ingredient, only the inhibitor, only the promoter, or both the inhibitor and the promoter. Further, for example, the ALS therapeutic composition of the present invention may be composed of only the active ingredient, or may contain the active ingredient and other additives to be described later.

The ALS therapeutic composition of the present invention preferably has, for example, the following compositions depending on a patient to be administered and treated.

(1) When a Patient has No ALS-Related Mutation for a Specific Gene

For a patient who has no specific mutation for the specific gene cluster (a C9orf72 gene, a SOD1 gene, a TBK1 gene, a TARDBP gene, a FUS gene and a NEK1 gene), the ALS therapeutic composition of the present invention preferably contains, for example, at least one of an inhibitor for the target A1 and a promoter for the target B1 as the active ingredient. The inhibitor for the target A1 and the promoter substance for the target B1 are as described above.

(2) When a Patient has an ALS-Related Mutation for a Specific Gene

For a patient who has a specific mutation for the specific gene cluster, the ALS therapeutic composition of the present invention preferably contains, for example, at least one of an inhibitor for the target A2 and a promoter for the target B2 as the active ingredient. The inhibitor for the target A2 and the promoter substance for the target B2 are as described above.

(3) When it is Unknown Whether or not a Patient has an ALS-Related Mutation for a Specific Gene

The ALS therapeutic composition of the present invention preferably contains, for example, at least one of an inhibitor for the target A3 and a promoter for the target B3 as the active ingredient. The ALS therapeutic composition having such a composition can be used, for example, for both a patient for whom presence or absence of the specific mutation for the specific gene group is known and a patient for whom presence or absence of the mutation is unknown. The inhibitor for the target A3 and the promoter substance for the target B3 are as described above.

The ALS therapeutic composition of the present invention may contain the active ingredient, and its composition is not particularly limited and can be appropriately set according to, for example, an administration method or the like. Regarding the ALS therapeutic composition of the present invention, an administration method, a dosage form and a form corresponding to the administration method, and the like are not particularly limited, and the description in the ALS treatment method of the present invention can be incorporated.

In the ALS therapeutic composition of the present invention, a content ratio of the active ingredient is not particularly limited. As described above, the ALS therapeutic composition of the present invention may contain an additive in addition to the active ingredient. The additive is preferably, for example, a pharmaceutically acceptable component, and can be appropriately determined according to an administration method, a subject to be administered, a dosage form, and the like.

Examples of the additive include a solvent, a diluent, an excipient, a carrier, and the like. Examples of the solvent and the diluent include liquid media such as an aqueous solvent, an alcohol solvent, a polyalcohol solvent, an oily solvent, and a mixed solvent thereof (for example, an emulsifying solvent). Examples of the aqueous solvent include water, physiological saline, isotonic solutions such as sodium chloride, and the like. Examples of the oily solvent include soybean oil, and the like. Examples of the excipient include lactose, starch, sucrose, and the like. In addition to these, examples of the additive include binders such as a starch paste, disintegrants such as starch and carbonate, lubricants such as talc and wax, and the like. Further, the ALS therapeutic composition of the present invention may contain, for example, a DDS agent for delivering the active ingredient to an affected area. The ALS therapeutic composition of the present invention may be, for example, a continuous release type composition in which the active ingredient is encapsulated in a carrier and the active ingredient is released over time. Examples of the carrier include polymer particles, and the like.

For example, in the case of the nucleic acid type expression inhibitor, the expression vector, and the like, the active ingredient may further contain a nucleic acid-introducing agent. Examples of the nucleic acid-introduction reagent include cationic lipids such as liposome, lipofectin, lipofectamine, DOGS (transfectum), DOPE, DOTAP, DDAB, DHDEAB, HDEAB, polybrene, poly (ethyleneimine) (PEI), and the like.

### ALS Diagnostic Method

As described above, the ALS diagnostic method of the present invention includes acquiring an expression level of a target A or a target B for a biological sample of a subject.

The target A is at least one gene selected from a group consisting of genes in Table 1-1 or a protein encoded by the gene.

When the expression level of the target A of the subject is higher than an expression level of the target A of a healthy subject, it is determined that the subject is suffering from ALS.

The target B is at least one gene selected from a group consisting of genes in Table 1-2 or a protein encoded by the gene.

When the expression level of the target B of the subject is lower than an expression level of the target B of a healthy subject, it is determined that the subject is suffering from ALS.

As described above, as a result of intensive studies, the inventors of the present invention have found that since there are targets with high expression and targets with low expression in ALS patients, by using expression levels of these targets as indicators for evaluation, whether or not a subject is suffering from ALS can be determined. The present invention is characterized in that an expression level of at least one of the target A and the target B is acquired for a biological sample of a subject and whether or not the subject is suffering from ALS is determined based on the expression level, and other constitutions and conditions are not particularly limited.

For the target A and the target B in the present invention, the description in the ALS treatment method of the present invention can be incorporated.

In the ALS diagnostic method of the present invention, in the acquisition, the expression level of the target A may be acquired, the expression level of the target B may be acquired, or the expression levels of both may be acquired. In the ALS diagnostic method of the present invention, information about at least one of the expression level of the target A and the expression level of the target B may be acquired. For example, measuring the expression level for a biological sample collected from the subject may be further included. Information about an already measured expression level may be used.

The type of the biological sample is not particularly limited, and is, for example, a sample collected from the brain, and, as a specific example, is a cerebrospinal fluid.

A method for measuring the expression level of the target A or the target B using the biological sample is not particularly limited. As described above, the target A and the target B may each be a gene or a protein encoded by the gene. Therefore, when the target is a gene, known methods used for gene expression analysis can be used, and when the target is a protein, known methods used for protein expression analysis can be used. For the expression of the gene, for example, quantitative PCR, microarray, RNA sequence, and the like can be used, and for the expression of the protein, for example, ELISA, Western blotting, mass spectrometry, and the like can be used.

The ALS diagnostic method of the present invention can determine whether or not a subject is suffering from ALS by comparing an expression level of the subject with an evaluation criterion. In the present invention, whether or not a subject is suffering from ALS includes meanings of, for example, whether or not the subject is actually suffering from ALS, whether or not there is a possibility that the subject is suffering from ALS, and whether or not there is a possibility for the subject to be suffering from ALS.

Examples of the evaluation criterion include the evaluation criteria C1, C2, C3, C4 and the like exemplified in the treatment method of the present invention.

The evaluation criterion C1 is an expression level of the target A in a healthy subject, and specifically, an expression level of the target A in a biological sample of the healthy subject. In the ALS diagnostic method of the present invention, when the expression level of the target A of the subject is higher than the evaluation criterion C1, it can be determined that the subject is suffering from ALS.

The evaluation criterion C2 is an expression level of the target A in an ALS patient, and specifically, an expression level of the target A in a biological sample of the ALS patient. In the ALS diagnostic method of the present invention, when the expression level of the target A of the subject is not significantly different or is high with respect to the evaluation criterion C2, it can be determined that the subject is suffering from ALS.

The evaluation criterion C3 is an expression level of the target B in a healthy subject, and specifically, an expression level of the target B in a biological sample of the healthy subject. In the ALS diagnostic method of the present invention, when the expression level of the target B of the subject is lower than the evaluation criterion C3, it can be determined that the subject is suffering from ALS.

The evaluation criterion C4 is an expression level of the target B in an ALS patient, and specifically, an expression level of the target B in a biological sample of the ALS patient. In the ALS diagnostic method of the present invention, when the expression level of the target B of the subject is not significantly different or is low with respect to the evaluation criterion C4, it can be determined that the subject is suffering from ALS.

Further, it is also possible that, as information about a biological sample of the patient, for example, information about both the target A and the target B is acquired, and, from a comparison with at least one of the evaluation criteria C1 and C2 and a comparison with at least one of the evaluation criteria C3 and C4, whether or not the subject is suffering from ALS is determined.

In the determination, the expression level of the target A may be, for example, the expression level of any one of the multiple targets described above, or the expression level of two or more of the targets. Similarly, the expression level of the target B may be, for example, the expression level of any one of the multiple targets described above, or the expression level of two or more of the targets.

For the expression level of the healthy subject and the expression level of the ALS patient, which are the evaluation criteria, for example, information acquired in advance from the healthy subject and the ALS patient can be used. Further, the expression level of the subject and the expression level of the healthy subject and the ALS patient that are used as the evaluation criteria are preferably, for example, the expression levels of the same type obtained from biological samples of the same type. The biological samples are each, for example, a sample derived from the brain, preferably, a cerebrospinal fluid.

In the ALS diagnostic method of the present invention, for example, by further selecting the types of the target A and the target B to be evaluated, the type of ALS can be determined in more detail for the subject. The forms exemplified below can each be said to be, for example, a test method or a classification method for ALS types.

(1) No Mutation in a Specific Gene Involved in ALS

As described above, it is known that some ALS patients are classified into a group that shows a specific mutation for the C9orf72 gene. As a result of further studies, the present inventors have found that when an ALS patient does not have the specific mutation for the C9orf72 gene, among the targets A, expression of the following target A4 is high, and among the targets B, expression of the following target B4 is low. Therefore, regarding the target A4, when an expression level of a subject is higher than the evaluation criterion C1 (expression level of a healthy subject), or regarding the target B4, when the expression level of the subject is lower than the evaluation criterion C3 (expression level of a healthy subject), it can be determined that the subject is a patient that is suffering from ALS and does not have the specific mutation for the C9orf72 gene.

Further, regarding the target A4, when an expression level of a subject is not significantly different or is high with respect to the evaluation criterion C2 (expression level of an ALS patient), or when the expression level of the subject is not significantly different or is low with respect to the evaluation criterion C4 (expression level of an ALS patient), it can be determined that the subject is a patient that is suffering from ALS and does not have a specific mutation for the C9orf72 gene.

The target A4 is, for example, at least one gene selected from a group consisting of the genes in Table 5-1 below (hereinafter, also referred to as a gene A4), or a protein encoded by the gene (hereinafter, also referred to as a protein A4). Further, the target B4 is, for example, at least one gene selected from a group consisting of the genes in Table 5-2 below (hereinafter, also referred to as a gene B4), or a protein encoded by the gene (hereinafter, also referred to as a protein B4).

TABLE 5-1
target A4 of ALS
(only C9orf72_wt)

HSPA8
PEBP1
HSP90AA1
NEFH
HSP90AB1
NDUFC2
PRKG1
ATP5MC3
C18orf32
SPARCL1
ERAP1
ARL17B
PDE5A
MIA2
CPQ
HHIP

TABLE 5-2
target B4 of ALS
(only C9orf72_wt)

ITPR2
LRRC4C
HRH3
LENG8
ACAP3
TNNI3K
GIT1
AP1G2
ADAP1
DDX51

(2) There is a Mutation in a Specific Gene Involved in ALS

As a result of further studies, the present inventors have found that when an ALS patient has the specific mutation for the C9orf72 gene, among the targets A, expression of the following target A5 is high, and among the targets B, expression of the following target B5 is low. Therefore, regarding the target A5, when an expression level of a subject is higher than the evaluation criterion C1 (expression level of a healthy subject), or regarding the target B5, when the expression level of the subject is lower than the evaluation criterion C3 (expression level of a healthy subject), it can be determined that the subject is a patient that is suffering from ALS and has the specific mutation for the C9orf72 gene.

Further, regarding the target A5, when an expression level of a subject is not significantly different or is high with respect to the evaluation criterion C2 (expression level of an ALS patient), or when the expression level of the subject is not significantly different or is low with respect to the evaluation criterion C4 (expression level of an ALS patient), it can be determined that the subject is a patient that is suffering from ALS and has a specific mutation for the C9orf72 gene.

The target A5 is, for example, at least one gene selected from a group consisting of the genes in Table 6-1 below (hereinafter, also referred to as a gene A5), or a protein encoded by the gene (hereinafter, also referred to as a protein A5). Further, the target B5 is, for example, at least one gene selected from a group consisting of the genes in Table 6-2 below (hereinafter, also referred to as a gene B5), or a protein encoded by the gene (hereinafter, also referred to as a protein B5).

TABLE 6-1
target A5 of ALS
(only C9orf72_mt)

FTL
MT3
MSH4
CHORDC1
IMMP2L
HES4
HIBCH
ADAM12
C12orf4
ATG4C
GREM2
SNCA
SKAP2
TCHP
MBP

TABLE 6-2
target B5 of ALS
(only C9orf72_mt)

CSNKA2IP
ROBO2
RGS7
SGCD
MPPED2
GABRG3
CLEC16A
GPC6
PLXDC2
DPH6
PDE10A
CARS1
TTYH1
GLIS3
CAMK4
VWC2

For the same reason as the above-described therapeutic method of the present invention, the genes included in the gene modules 2, 5 and 11 shown in Table 8 above are presumed to be useful as targets for the diagnosis of ALS.

### Test Method for ALS Susceptibility

The ALS diagnostic method of the present invention can also be used, for example, as a method performed by a person other than a doctor. In this case, the present invention is a method for testing a possibility of suffering from ALS, and includes acquiring an expression level of the target A or the target B for a biological sample of a subject. When the expression level of the target A of the subject is higher than an expression level of target A of a healthy subject, it is an inventor indicating a possibility that the subject is suffering from ALS. When the expression level of the target B of the subject is lower than an expression level of the target B of a healthy subject, it is an indicator indicating a possibility that the subject is suffering from ALS. The test method for ALS susceptibility of the present invention can incorporate, for example, the diagnostic method of the present invention.

In the test method of the present invention, for example, regarding a biological sample of the subject, expression of a target A4 among the targets A or expression of a target B4 among the targets B is acquired. The target A4 is at least one gene selected from a group consisting of the genes in Table 5-1 or a protein encoded by the gene. The target B4 is at least one gene selected from a group consisting of the genes in Table 5-2 or a protein encoded by the gene. When the expression level of the target A4 of the subject is, for example, higher than an expression level of the target A4 of a healthy subject, it is an indicator indicating a possibility that the subject is suffering from ALS and is of a type having no ALS-related mutation for the C9orf72 gene. When the expression level of the target B4 of the subject is, for example, lower than an expression level of the target B4 of a healthy subject, it is an indicator indicating a possibility that the subject is suffering from ALS and is of a type having no ALS-related mutation for the C9orf72 gene.

In the test method of the present invention, for example, regarding a biological sample of the subject, expression of a target A5 among the targets A or expression of a target B5 among the targets B is acquired. The target A5 is at least one gene selected from a group consisting of the genes in Table 6-1 or a protein encoded by the gene. The target B5 is at least one gene selected from a group consisting of the genes in Table 6-2 or a protein encoded by the gene. When the expression level of the target A5 of the subject is, for example, higher than an expression level of the target A5 of a healthy subject, it is an indicator indicating a possibility that the subject is suffering from ALS and is of a type having an ALS-related mutation for the C9orf72 gene. When the expression level of the target B5 of the subject is, for example, lower than an expression level of the target B5 of a healthy subject, it is an indicator indicating a possibility that the subject is suffering from ALS and is of a type having an ALS-related mutation for the C9orf72 gene.

### Screening Method

The present invention provides a method for screening a therapeutic agent for ALS, which includes: evaluating an inhibitory ability against the target A using a candidate substance, or evaluating a promoting ability for the target B using a candidate substance; and selecting a candidate substance exhibiting the inhibitory ability or a candidate substance exhibiting the promoting ability as a therapeutic agent for ALS. In the present invention, the target A and the target B can incorporate the above descriptions.

When the target A is at least one gene selected from a group consisting of the genes in Table 1-1, in the evaluation, for example, the candidate substance and the gene are allowed to coexist, and the expression of the gene is measured. When the expression level of the gene is lower than an expression level of the gene in the absence of the candidate substance, it is evaluated that the candidate substance has an inhibitory ability to inhibit the expression of the gene. The lower the expression level in the case of coexistence is relative to the expression level of the gene in the case of absence, the stronger the inhibitory ability of the candidate substance can be relatively evaluated, for example.

When the target A is a protein encoded by at least one gene selected from a group consisting of the genes in Table 1-1, in the evaluation, for example, the candidate substance and the protein are allowed to coexist, and a function of the protein is measured. When a degree of the function of the protein is lower than a degree of the function of the protein in the absence of the candidate substance, it is evaluated that the candidate substance has a inhibitory ability to inhibit the function of the protein. The lower the degree of the function in the case of coexistence is relative to the degree of the function of the protein in the case of absence, the stronger the inhibitory ability of the candidate substance can be relatively evaluated, for example.

When the target B is at least one gene selected from a group consisting of the genes in Table 1-2, in the evaluation, for example, the candidate substance and the gene are allowed to coexist, and the expression of the gene is measured. When the expression level of the gene is higher than an expression level of the gene in the absence of the candidate substance, it is evaluated that the candidate substance has promoting ability to promote the expression of the gene. The higher the expression level in the case of coexistence is relative to the expression level of the gene in the case of absence, the stronger the promoting ability of the candidate substance can be relatively evaluated, for example.

When the target B is a protein encoded by at least one gene selected from a group consisting of the genes in Table 1-2, in the evaluation, for example, the candidate substance and the protein are allowed to coexist, and a function of the protein is measured. When a degree of the function of the protein is higher than a degree of the function of the protein in the absence of the candidate substance, it is evaluated that the candidate substance has an promoting ability to promote the function of the protein. The higher the degree of the function in the case of coexistence is relative to the degree of the function of the protein in the case of absence, the stronger the promoting ability of the candidate substance can be relatively evaluated, for example.

The type of the candidate substance is not particularly limited, and examples thereof include low molecular weight compounds, nucleic acids, proteins, peptides, and the like. Examples of the nucleic acids include candidate substances randomly designed from basic structures such as an interfering nucleic acid, an antisense, an antigene, and a ribozyme, as described above. Examples of the proteins include candidate substances randomly designed from basic structures of antibodies, and examples of the peptides include candidate substances randomly designed from basic structures of antibodies and antigen-binding fragments.

### EXAMPLES

Next, Examples of the present invention are described. However, the present invention is not limited by the following Examples. Commercially available reagents were used based on protocols thereof unless otherwise indicated.

### Example A

The primary motor vision (M1) of the cerebral cortex was collected from each of frozen postmortem brains of sporadic ALS patients without ALS-specific mutations in C9orf72 (n=17; hereinafter, sALS), ALS patients with ALS-specific mutations in C9orf72 (n=6; hereinafter, c9ALS), and healthy subjects without ALS (n=17), which were matched in gender and age. The sALS was a group without mutations in SOD1, TARDBP, FUS, NEK1, GRN, MAPT, and TBK1 in addition to C9orf72. The c9ALS was a group including sporadic ALS patients and familial ALS patients with ALS-specific mutations in C9orf72. Then, nuclei were extracted from the collected tissues, and single-cell RNA analysis was performed using the extracted samples. Sample preparation and RNA analysis were performed using a method of Example B to be described later.

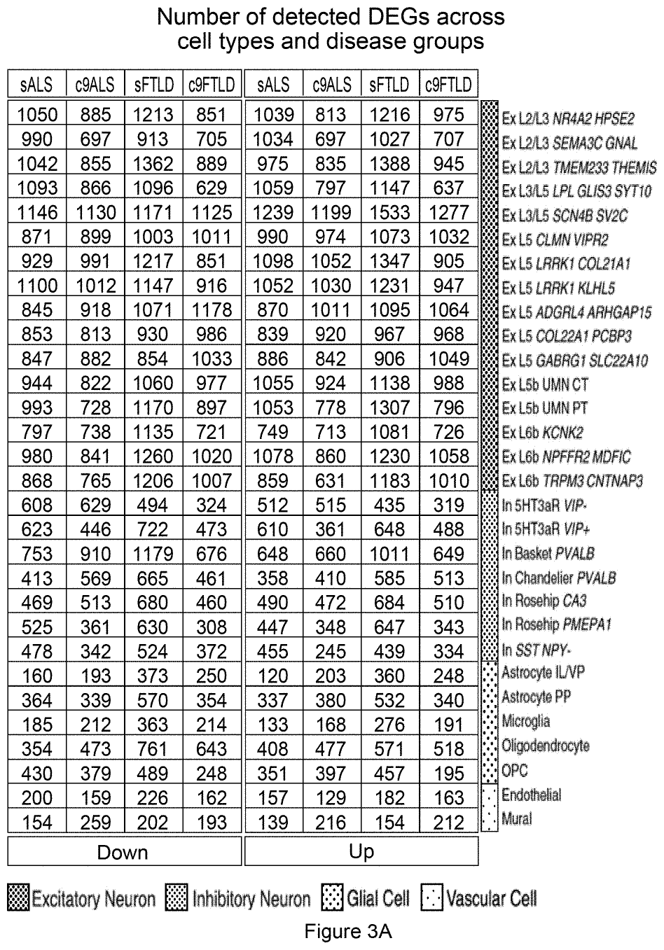

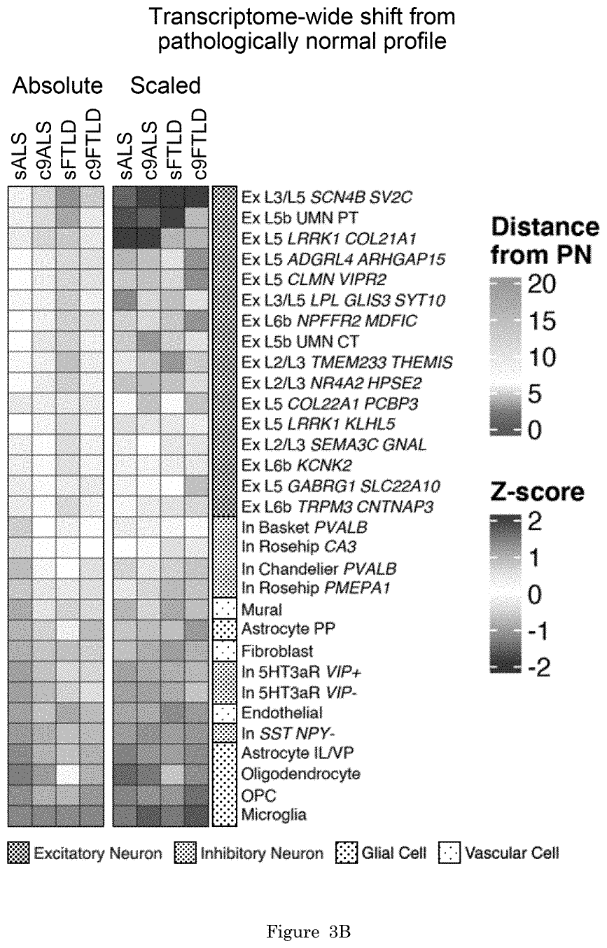

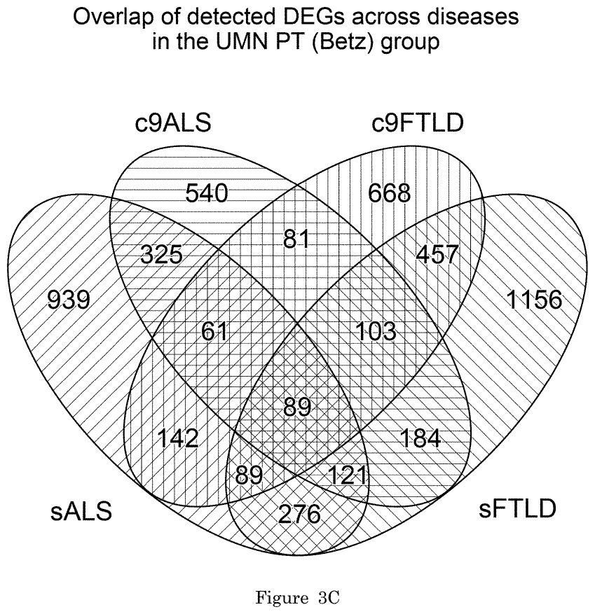

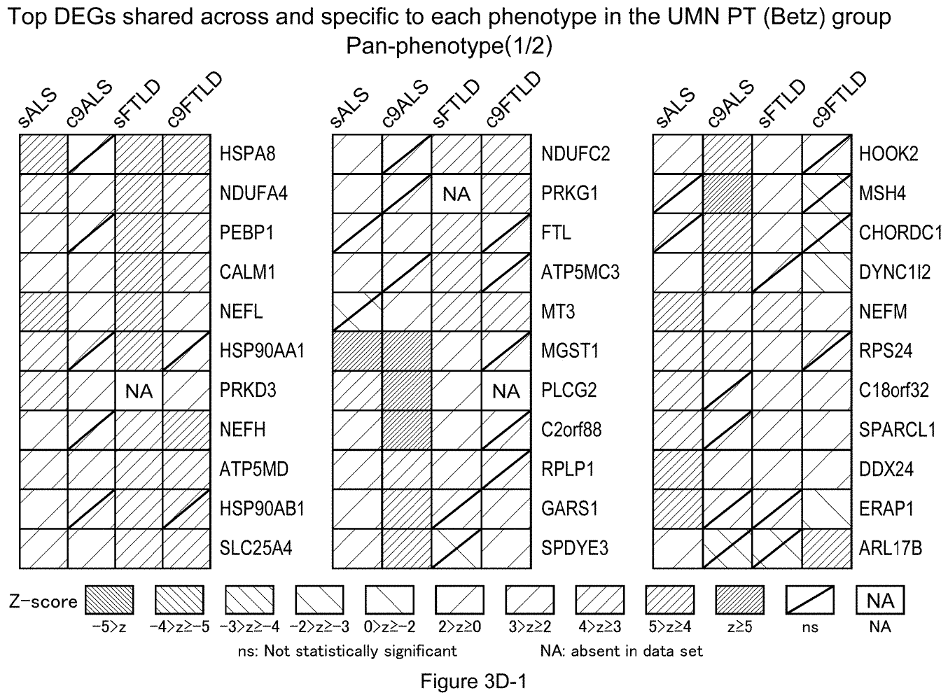

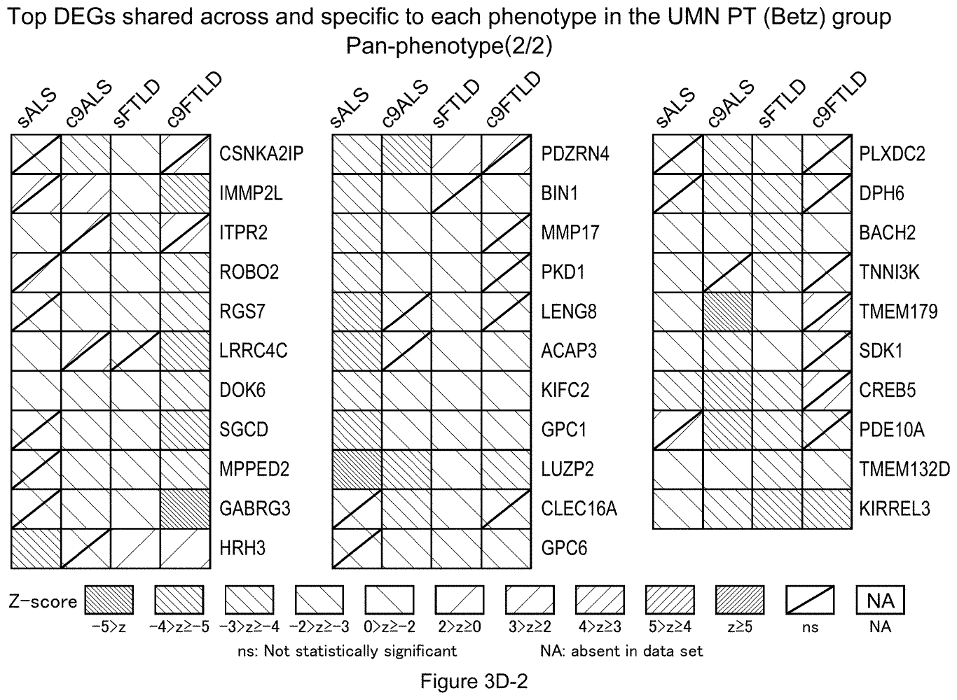

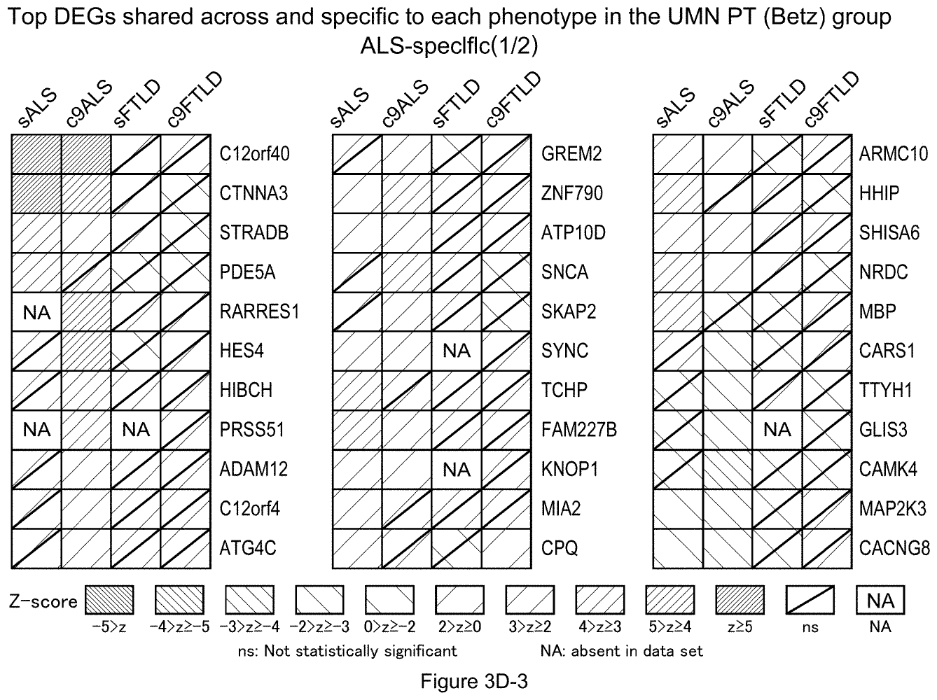

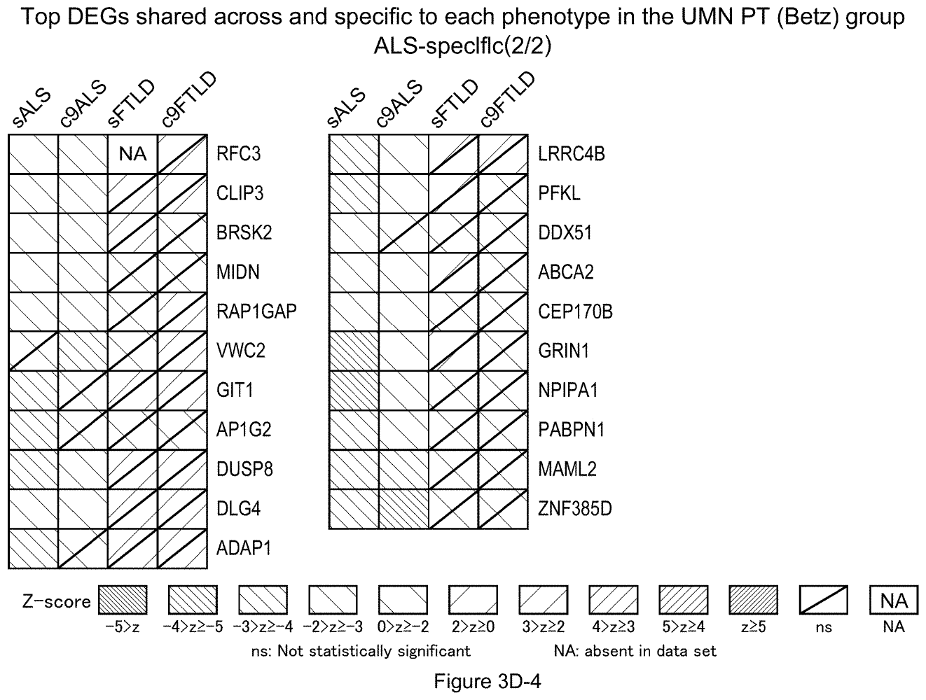

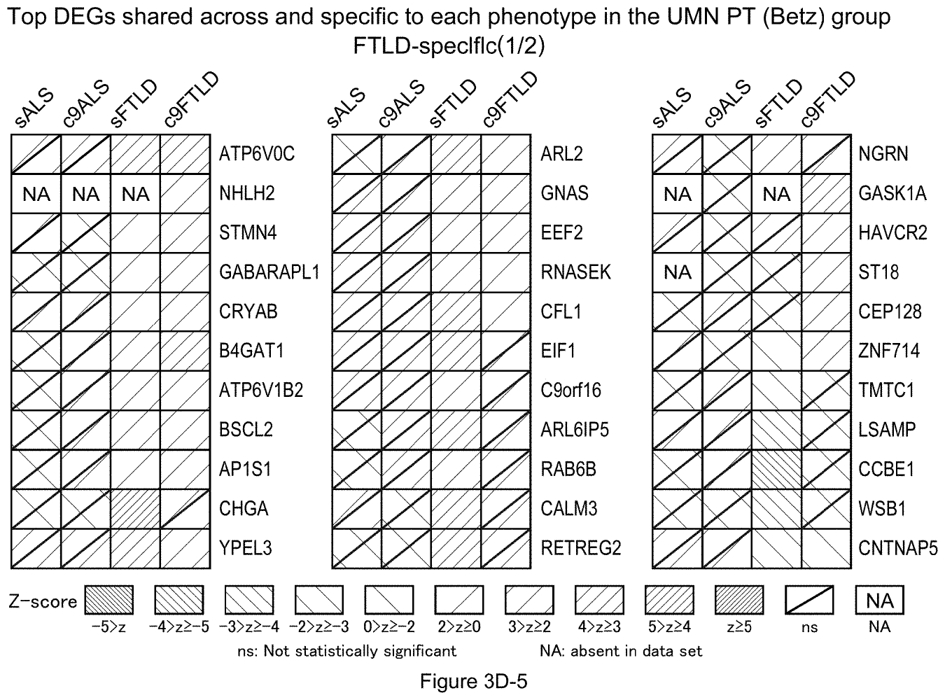

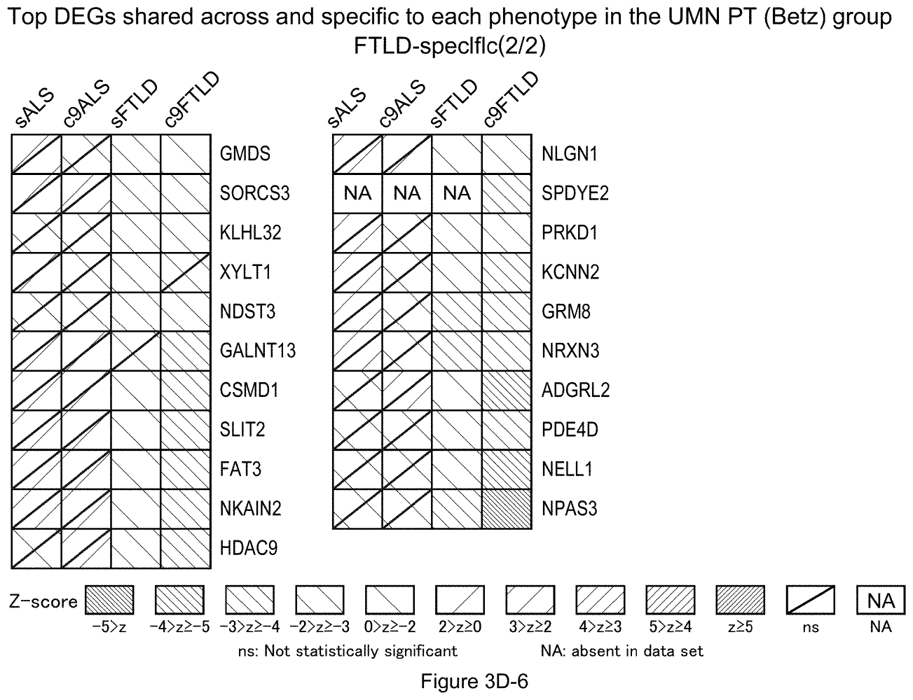

Then, based on expression levels in healthy subjects, differentially expressed genes (DEGs) of which expression was significantly increased or decreased in sALS or c9ALS were extracted. These results are shown in FIG. 3D. FIG. 3D shows expression levels of DEGs of which expression was significantly increased or decreased in sALS or c9ALS with a heat map of Z-scores (p<0.005). As shown in FIG. 3D, target genes of which expression was significantly increased or decreased in sALS and c9ALS were clarified.

Specifically, in FIG. 3D, the genes of which expression was significantly increased in sALS or c9ALS are the genes in Table 1-1 above, and the genes of which expression was significantly decreased are the genes in Table 1-2. Therefore, it is clear that ALS can be treated by inhibiting expression of the genes in Table 1-1 above or functions of proteins encoded by the genes, or by promoting expression of the genes in Table 1-2 above or functions of proteins encoded by the genes, and it is clear that ALS can be diagnosed by measuring expression levels of these.

Further, in FIG. 3D, genes of which expression was significantly increased only in sALS without ALS-specific mutations in C9orf72 not in ALS with ALS-specific mutations in C9orf72 are the genes in Table 2-1 above, and genes of which expression was significantly decreased are the genes in Table 2-2 above. Therefore, it is clear that, among ALS, sALS without ALS-specific mutations in C9orf72 can be selectively treated by inhibiting or promoting expression of these genes or functions of proteins encoded by the genes, and it is clear that diagnosis for the classification of the sALS can be performed by measuring expression levels of these.

Further, in FIG. 3D, genes of which expression was significantly increased only in ALS with ALS-specific mutations in C9orf72 not in sALS without ALS-specific mutations in C9orf72 are the genes in Table 3-1 above, and genes of which expression was significantly decreased are the genes in Table 3-2 above. Therefore, it is clear that, among ALS, ALS without ALS-specific mutations in C9orf72 can be selectively treated by inhibiting or promoting expression of these genes or functions of proteins encoded by the genes, and it is clear that diagnosis for the classification of the ALS can be performed by measuring expression levels of these.

Further, in FIG. 3D, genes of which expression was significantly increased regardless of whether or not there are ALS-specific mutations in C9orf72 are the genes in Table 4-1 above, and genes of which expression was significantly decreased are the genes in Table 4-2 above. Therefore, it is clear that ALS with or without ALS-specific mutations in C9orf72 can be treated by inhibiting or promoting expression of these genes or functions of proteins encoded by the genes, and it is clear that diagnosis for the classification of the ALS can be performed by measuring expression levels of these.

Further, in FIG. 3D, genes of which expression was significantly increased only in sALS without ALS-specific mutations in C9orf72 among sALS are the genes in Table 5-1 above, and genes of which expression was significantly decreased are the genes in Table 5-2 above. Therefore, it is clear that, by inhibiting or promoting expression of these genes or functions of proteins encoded by the genes, diagnosis for the classification of sALS without ALS-specific mutations in C9orf72 among ALS can be performed.

Further, in FIG. 3D, genes of which expression was significantly increased only in ALS with ALS-specific mutations in C9orf72 are the genes in Table 6-1 above, and genes of which expression was significantly decreased are the genes in Table 6-2 above. Therefore, it is clear that, by inhibiting or promoting expression of these genes or functions of proteins encoded by the genes, diagnosis for the classification of ALS with ALS-specific mutations in C9orf72 among ALS can be performed.

### Example B

### Methods

Human Sample Selection and Preparation

Human tissue analysis was conducted as exempt human research, considering frozen post-mortem brain samples obtained from the Neuropathology Laboratory at the Mayo Clinic (Jacksonville, Fla. USA) were not specifically collected for this study. All cases were carefully analyzed by experienced and certified neuropathologists. TDP-43 pathology was confirmed in all ALS and FTLD samples based upon current consensus criteria, which investigates cortical and subcortical distribution of TDP-43 neuropathologic inclusions. A section of the cerebellum was screened for C9ORF72-related pathology with P62 immunohistochemistry. For all C9orf72-associated cases, repeat expansions were confirmed via Southern blot. Extent of upper and lower motor neuron involvement was investigated in all samples using Luxol fast blue and IBA-1 immunohistochemistry in the motor cortex, midbrain, and medulla. Motor neuron degeneration was confirmed in all ALS cases and found absent from all FTLD and control samples selected.

The inventors selected a total of 64 age- and sex-matched individuals with sporadic ALS (sALS; n=17), C9orf72-associated ALS (c9ALS; n=6), sporadic FTLD (sFTLD; n=13), C9orf72-associated FTLD (c9FTLD; n=11), or found pathologically normal (n=17). Each cohort included similar numbers of male and female samples (sALS 8:9; c9ALS 3:3; sFTLD 7:6; c9FTLD 7:4; PN 8:9). Each C9orf72-associated disease cohort included patients with a positive family history of either ALS or FTLD. All other disease cases selected were considered sporadic, which is representative of the majority of patients: no family history and no defined genetic risk factor (no mutation in SOD1, TARDBP, FUS, C9orf72, NEK1, GRN, MAPT, or TBK1). For each brain selected, approximately 300 mg of the primary motor cortex was dissected by the Mayo Clinic Neuropathology Laboratory.

Isolation of Nuclei from Post-Mortem Frozen Human Brain Tissue for Single Nuclear RNA-Sequencing

Nuclei isolation protocol was adapted from Lee et al (Cell Type-Specific Transcriptomics Reveals that Mutant Huntingtin Leads to Mitochondrial RNA Release and Neuronal Innate Immune Activation. Neuron 107, 891-908.e8 (2020).). All procedures were performed on ice. Tissue was homogenized in 700 μL of homogenization buffer (320 mM sucrose, 5 mM CaCl2, 3 mM Mg(CH₃COO)₂, 10 mM Tris HCl [pH 7.8], 0.1 mM EDTA [pH 8.0], 0.1% NP-40, 1 mM β-mercaptoethanol, and 0.4 U/μL SUPERaseIn RNase Inhibitor (ThermoFisher Scientific, Waltham Mass.) with a 2 mL KIMBLE Dounce tissue grinder (MilliporeSigma, Burlington Mass.) using 10 strokes with loose pestle followed by 10 strokes with tight pestle.

From the mouse tissue samples the entire striatum was homogenized. From the human tissue samples 100 mg of grey matter was sectioned and homogenized. Homogenized tissue was filtered through a 40 μm cell strainer and mixed with 450 μL of working solution (50% OptiPrep density gradient medium (MilliporeSigma, Burlington Mass.), 5 mM CaCl₂, 3 mM Mg(CH₃COO)₂, 10 mM Tris HCl [pH 7.8], 0.1 mM EDTA [pH 8.0], and 1 mM (3-mercaptoethanol). The mixture was then slowly pipetted onto the top of an OptiPrep density gradient containing 750 μL of 30% OptiPrep Solution (134 mM sucrose, 5 mM CaCl₂, 3 mM Mg(CH₃COO)₂, 10 mM Tris HCl [pH 7.8], 0.1 mM EDTA [pH 8.0], 1 mM β-mercaptoethanol, 0.04% NP-40, and 0.17 U/μL SUPERase In RNase Inhibitor) on top of 300 μL of 40% OptiPrep Solution (96 mM sucrose, 5 mM CaCl₂, 3 mM Mg(CH₃COO)₂, 10 mM Tris HCl [pH 7.8], 0.1 mM EDTA [pH 8.0], 1 mM (3-mercaptoethanol, 0.03% NP-40, and 0.12 U/μL SUPERase In RNase Inhibitor) inside a Sorenson Dolphin microcentrifuge tube (MilliporeSigma, Burlington Mass.). Nuclei were pelleted at the interface of the OptiPrep density gradient by centrifugation at 10,000×g for 5 min at 4° C. using a fixed angle rotor (FA-45-24-11-Kit). The nuclear pellet was collected by aspirating ˜100 μL from the interface and transferring to a 2.5 mL Eppendorf tube. The pellet was washed with 2% BSA (in 1×PBS) containing 0.12 U/μL SUPERase In RNase Inhibitor. The nuclei were pelleted by centrifugation at 300×g for 3 min at 4° C. using a swing-bucket rotor (S-24-11-AT). Nuclei were washed three times with 2% BSA and centrifuged under the same conditions. The nuclear pellet was resuspended in 100 μL of 2% BSA.

### Sequencing Data Preprocessing and Analysis

Droplet-based snRNA sequencing libraries were prepared using the Chromium Single Cell 3′ Reagent Kit v3 (10× Genomics, Pleasanton Calif.) according to the manufacturer's protocol and sequenced on a NovaSeq 6000 at the Broad Institute Genomics Platform. FASTQ files were aligned to the pre-mRNA annotated human reference genome GRCh38. Cell Ranger v4.0 (10× Genomics, Pleasanton Calif.) was used for genome alignment and feature-barcode matrix generation.

The inventors used the ACTIONet and scran R packages to normalize, batch correct, and cluster single-cell gene counts. A curated set of known cell type-specific markers was used to annotate individual cells with their expected cell type and assign a confidence score to each annotation. The inventors removed cells with high mitochondrial RNA content, abnormally low or high RNA content (relative to the distribution of its specific cluster), ambiguous overlapping profiles resembling dissimilar cell types (generally corresponding to doublet nuclei), and cells corresponding to graph nodes with a low k-core or low centrality in the network (generally corresponding to high ambient RNA content or doublet nuclei).

Cell type-specific pseudo-bulk differential gene expression (DGE) analysis was performed using ACTIONet and limma for sufficiently abundant cell types using age, sex, and disease group as design covariates and gene-wise single-cell-level variance as weights for the linear model. The inventors found Braak stage to be a poor predictor of gene expression and omitted it from the design formula. Genes were considered differentially expressed if they had an FDR-corrected p-value <0.005 and an absolute log 2-fold change >1 standard deviation for that cell type relative to the pathologically normal control group. To ensure that DGE results were reproducible and robust to differences in cell type abundance, the inventors sampled with replacement equal numbers of individuals and cells per individual for each cell type and repeated the pseudo-bulk analysis. Lastly, the inventors repeated the analyses using DESeq2 as the model-fitting algorithm in lieu of limma to ensure replicability across methods. In all cases DGE results were consistent and the inventors used the pseudo-bulk limma results for all downstream analyses. Pathway analysis was performed using the g:Profiler R package.

### WGCNA

The inventors used R package WGCNA to perform the weighted correlation network analysis on pseudo-bulk expression profiles. A signed network was constructed with the UMN PT and L3/L5 LR clusters shown in the ACTIONet plot of excitatory neurons. The co-expression similarity was raised to the power B=8 to arrive at the network adjacency (R2 is 0.8 when β=8). The modules were identified using the R function blockwiseConsensusModules with minModuleSize=30. Hub genes (genes with highest module membership) in each consensus module were identified using the R function signedKME. Pathway analysis was performed using the R package gprofiler2 considering only protein-coding hub genes with kME value >0.6.

### Immunofluorescent Labeling and Confocal Microscopy

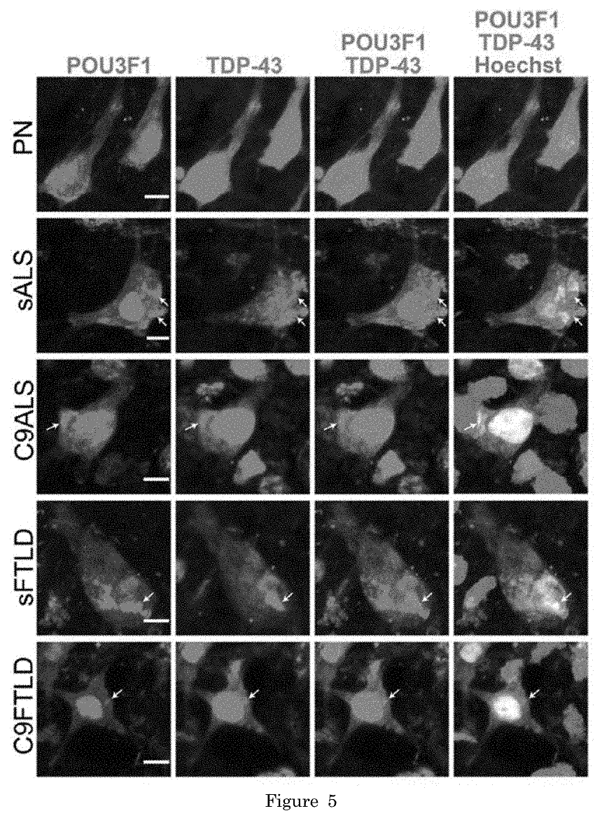

Patient tissues were embedded in Allan Scientific™ Neg-50™ Frozen Section Medium (Thermo Scientific #6502), cryosectioned to 20 μm thick, and transferred to glass slides (VWR #48311-703). Next, the tissues were subject to 100% ice-cold acetone (Sigma-Aldrich #320110) for 10 minutes followed by 15 minutes of trisbuffered saline with 0.25% Triton X-100 then blocked for 1 hour in tris-buffered saline with Tween 20 solution at 0.05% (BioRad #161-0781) supplemented with sterile 2% Normal Donkey serum (Abcam #ab138579) and 0.1% fish gelatin (Sigma-Aldrich #G7765). Next, the slides were incubated overnight at 4 degrees Celsius in a slide humidity chamber with primary antibodies POU3F1 (Millipore Sigma MABN738) and TDP-43 (ProteinTech 10782-2-AP) or CRYM (Abcam ab220085). Next, the slides were washed and incubated with Donkey anti-Mouse IgG (H+L) 488 nm and Donkey anti-Rabbit IgG (H+L) 546 nm (ThermoFisher #A-21202, A-10040, respectively) for FIG. 5A. The slides were washed and incubated with Donkey anti-Mouse IgG (H+L) 488 nm and Donkey anti-Rabbit IgG (H+L) 647 nm (ThermoFisher #A-21202, #A-31573, respectively) for FIG. 5B. Following additional washes, a 10 mg/mL stock of Hoechst 33342, Trihydrochloride, Trihydrate (ThermoFisher #H1399) was used at 1 μL per 10 mL of washing solution for 10 minutes. Next, the tissues were treated with a solution containing TrueBlack (Biotium) at 50 μL per 1 mL of 70% ethanol for 10 seconds, and then washed with tris-buffered saline solution (no detergent). Tissues were then mounted using ProLong™ Gold Antifade Mountant (ThermoFisher #P36930). Mounted slides were imaged on a Zeiss Observer.Z1 LSM 700 confocal microscope (Carl Zeiss AG, Oberkochen, Germany) using a EC Plan-Neofluar 40X/1.30 Oil DIC M27 objective. Z-stack images were max-projected using Fiji.

### Results

### Characterization of Human Primary Motor Cortex

The inventors first sought to characterize the diversity of cell types and marker genes for both neuronal and non-neuronal cells in the human primary motor cortex. After applying stringent quality control metrics and cell filtering, the inventors report 380,610 single-nucleus profiles across 64 primary motor cortex samples, corresponding to ˜6000 postquality control nuclei per donor (see Methods). Compared to previous studies, the number of human cells captured represents a 4-fold increase in cell count, 10-fold increase in the number of individuals, and substantial increase in cell level resolution.

The inventors annotated 46 transcriptionally-distinct cell subpopulations, all of which were well-mixed and reproducible across individuals, sexes, genotypes, and phenotypes using our recently-developed single-cell data analysis toolkit, ACTIONet, and well-curated cell type-specific marker genes.

The inventors characterized 19 subtypes of excitatory neurons in six major groups. Translaminar pyramidal neurons spanning layers 2 through 5 formed the largest group, consisting of 6 subtypes based on layerspecific marker gene expression. These showed a gradient of layer-specific marker gene expression, with markers for adjacent layers enriched in adjacent regions of the cluster in a mostly linear fashion, consistent with reports of non-discrete transcriptional identity of translaminar cortical excitatory neurons in mice. No subtype or cluster showed exclusive enrichment for layer 4 markers, consistent with previous reports that layer 4 is absent from agranular primary motor cortex, but adjacent subpopulations with layer 3 and layer 5 markers (Ex L3/L5) and layer 5 subtypes (Ex L5) showed overlapping expression of layer 4 markers. The inventors also detected a distinct subtype of SCN4B+ cells expressing markers of both Layer 3 and Layer 5 at the layer 3/5 interface, which showed the highest expression of neurofilament genes NEFL, NEFM, and NEFH, indicative of large axon caliber neurons. This population likely corresponds to FEZF2-L3/L5 long-range projecting neurons, and is henceforth referred to as “L3/L5 LR”.

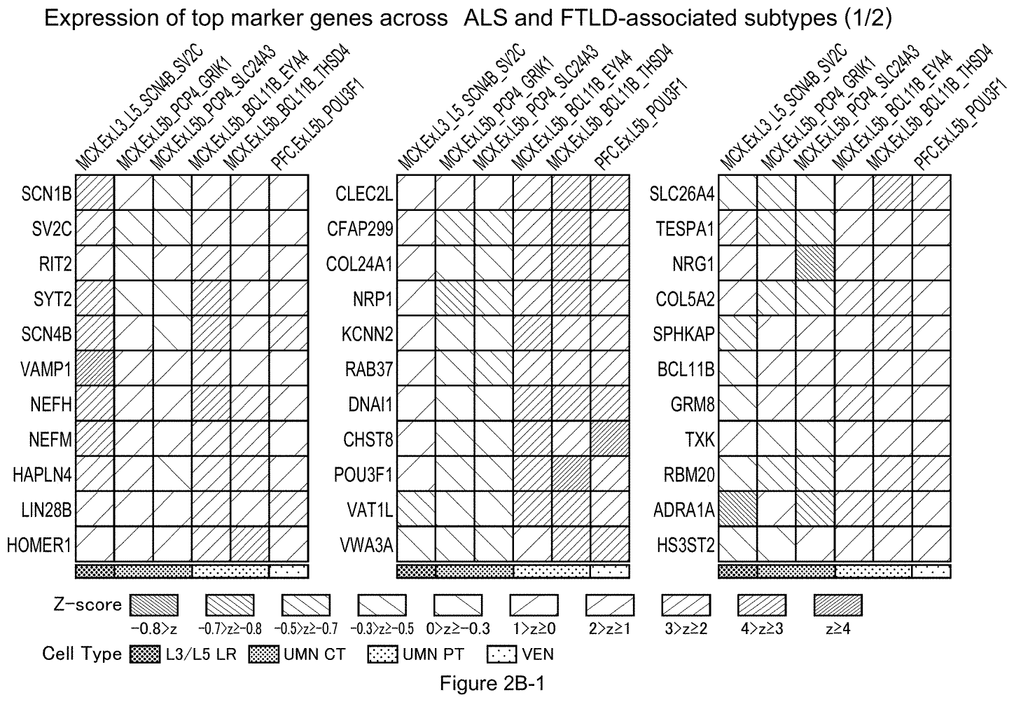

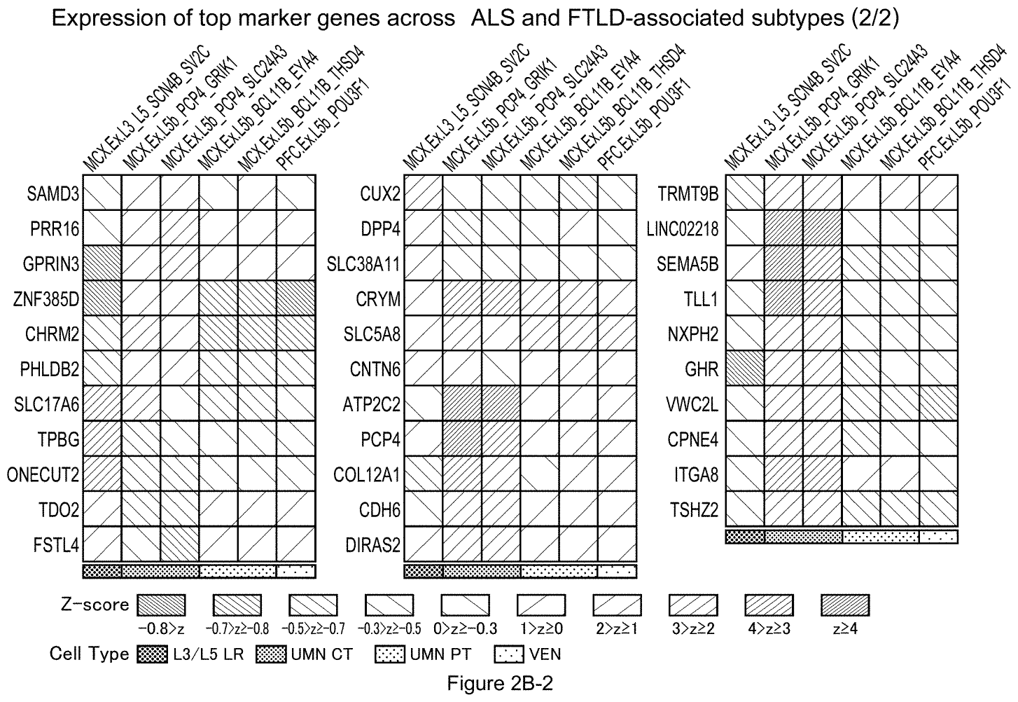

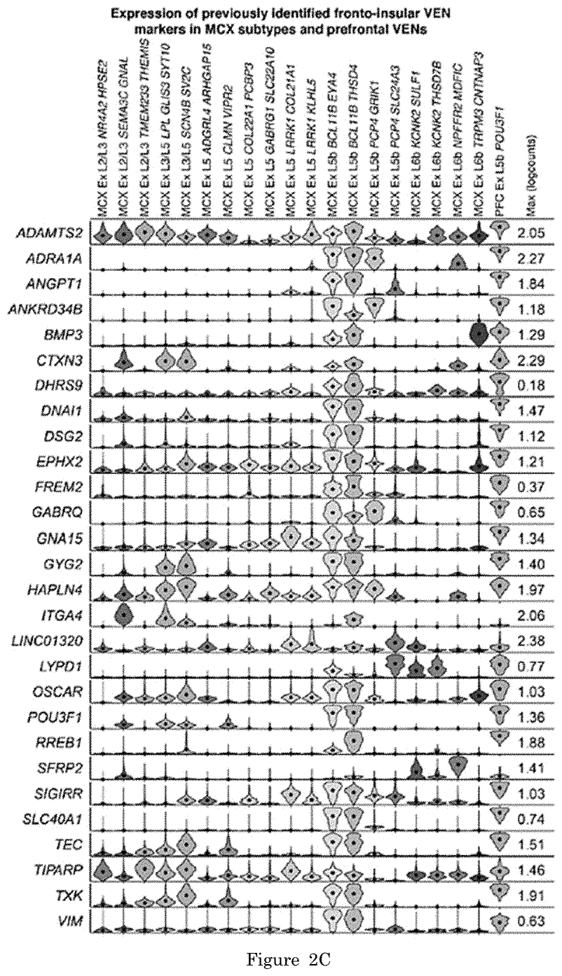

The inventors identified four distinct subtypes of FEZF2/CRYM+ layer 5b neurons partitioned into two groups. The smaller of these two groups corresponded to two transcriptionally distinct subtypes (BCL11B/EYA4 and BCL11B/THSD4) of giant pyramidal tract upper motor neurons (UMN PT), also known as Betz cells, which expressed higher levels of neurofilament gene NEFH, and highly-specific enrichment of the Betz and VEN identity marker POU3F1. These cells possessed the highest average RNA content among all cell types, indicating a large cell volume characteristic of giant Betz cells. Expression of key markers and expected Betz morphology was confirmed by immunofluorescence (IF) (FIG. 2A). These cells are of particular interest as they are known to be selectively vulnerable in ALS.

The larger group of the layer 5b FEZF2/CRYM+ cells (PCP4/GRIK1 and PCP4/SLC24A3) was also largely enriched for several canonical UMN markers but was reduced in or lacked expression of the long-range projection markers NEFH and SCN4B (FIGS. 2B-1 and 2B-2). Observation of these cells by IF (not shown) showed that they were also layer 5b pyramidal neurons, but appeared to be much smaller than Betz cells, leading us to conclude that these are likely the corticobulbar tract upper motor neurons (UMN CT) that innervate the cranial nerve nuclei, the reticular formation, and the red nucleus.

The inventors identified a group of four transcriptionally similar excitatory neuron subtypes that showed selective enrichment for markers of layer 6b, and human orthologs of mouse layer 6 subplate-derived deep cortical neurons36, as well as a cluster of layer 2/3 neurons with highly-specific expression of NR4A2.

The inventors captured 16 distinct populations of cortical inhibitory neurons, spanning multiple, highly-resolved subtypes of somatostatin-expressing GABAergic interneurons (NPY+ and NPY−), parvalbumin-expressing basket and chandelier cells, 5HT3aR-expressing interneurons (VIP+ and VIP−), and two populations of the recently characterized rosehip interneurons (CA3+ and PMEPA1+).

The inventors also recovered all expected classes of cortical glial, vascular, and immune cell types, including oligodendrocytes, oligodendrocyte progenitors, two subtypes (protoplasmic, interlaminar) of astrocytes, fibroblasts, arterial and venous subtypes of endothelial cells, smooth muscle and pericyte mural cells, and T cells.

### Motor Cortex Betz Cells are Transcriptional Analogues to Frontal and Temporal Cortex VENs

The inventors next asked whether our discovered clusters of Betz cells in the primary motor cortex share intrinsic molecular markers with VENs, motivated by the fact that: (1) Betz cells and VENs display enhanced vulnerability in ALS and FTLD respectively; (2) Betz cells are large extratelencephalic-projecting layer 5 neurons and VENs have been recently hypothesized to also be extratelencephalic-projecting layer 5 neurons.

To compare VENs and Betz cells at similar resolution, the inventors used data from our recent single-cell profiling of the dorsolateral PFC carried out in the context of schizophrenia, but focusing only on the subset of 24 pathologically normal individuals. Detailed clustering and annotation of 107,358 PFC excitatory neurons revealed a neurofilament-enriched layer 5b subtype with selective expression of POU3F1 and co-expression of numerous Betz cell markers, which the inventors determined to be the VEN population.

Pairwise transcriptome-wide analysis revealed that almost all annotated excitatory populations in the MCX could be mapped to a synonymous subtype in the dorsolateral PFC, and that both MCX Betz cell subtypes mapped most specifically to the PFC VEN subtype (r=0.57 and 0.61).

The inventors then identified and compared the top 50 marker genes of UMN, L3/L5 LR, and VEN subtypes and found that Betz cells and VENs possessed nearly identical expression patterns across these genes, and that a subset of these markers were also highly expressed in L3/L5 LR neurons, but very few were shared with corticobulbar motor neurons (FIGS. 2B-1 and 2B-2).

When the inventors compared our data with the VEN markers described in a recent, targeted single-cell study of frontal insula VENs27, the inventors again found that all reported markers, in addition to the previously described CTIP2/BCL11B and FEZF2, are also very specifically expressed in Betz cells and, to a lower extent, other layer 5 UMNs and L3/L5 LR cells. This analysis also confirmed the annotation of our PFC VEN cluster (FIG. 2C).

This finding reveals a close molecular similarity between the two cortical populations that display the most vulnerability in ALS and FTLD, and provides further evidence for the hypothesis that VENs are in fact extratelencephalic-projecting neurons.

### Cell Type-Specific Differential Gene Expression Analysis of ALS and FTLD

The inventors next performed cell type-specific pseudo-bulk differential expression analysis across all five phenotypes: sporadic ALS (sALS; n=17), C9orf72-associated ALS (c9ALS; n=6), sporadic FTLD (sFTLD; n=13), C9orf72-associated FTLD (c9FTLD; n=11), and pathologically normal (PN; n=17). To ensure that the inventors had sufficient power across all experimental groups for less abundant populations, the inventors aggregated highly similar subtypes within the same group for differential expression analysis (e.g. the inventors treated both transcriptional subtypes of Betz cells as a single population denoted as Ex UMN PT), and excluded In SST/NPY+ and T cells which were insufficiently abundant across donors and disease groups, and too dissimilar to aggregate with other subtypes.

Although large layer 5 projection neurons would be expected to be the most affected in both ALS and FTLD, the inventors expected to see alterations in other cortical layers as well due to spreading of cortical atrophy in late-stage disease. Indeed, the inventors observed a large disparity in the number of dysregulated genes between excitatory neurons and all other cell populations across all phenotypes and found that all excitatory neurons across cortical layers displayed severe transcriptional dysregulation (FIG. 3A, 3B).

The left figure (Absolute) in FIG. 3B shows distances of expression fluctuations of cell types for each disease group when compared to healthy subjects (PN) (Distance from PN). A larger distance means a larger expression fluctuation when transcriptome as a whole is analyzed. The right figure (Scaled) in FIG. 3B shows Z-Scores. It was found that, in each disease group, the absolute distance was small in Inhibitory Neuron, Glial Cell and Vascular Cell, whereas the absolute distance was large in Excitatory Neuron, that is, the expression fluctuation was large. In sALS, significant fluctuation was confirmed in Ex L3/L5 SCN4B SV2C, Ex L5b UMN PT, Ex L5 LRRK1 COL21A1, Ex L3/L5 LPL GLIS3 SYT10. In c9ALS, significant fluctuation was confirmed in Ex L3/L5 SCN4B SV2C, Ex L5b UMN PT, Ex L5 LRRK1 COL21A1, Ex L5b UMN CT. In sFTLD, particularly significant fluctuation was confirmed in Ex L3/L5 SCN4B SV2C, Ex L5b UMN PT. In c9FTLD, significant fluctuation was confirmed in Ex L3/L5 SCN4B SV2C, ExADGRL4 ARHGAP15, Ex L5 CLMN VIPR2, ExL6bNPFFR2 MDFIC.

Our analysis showed that a significant fraction of upregulated differentially expressed genes (DEGs) was shared across excitatory neurons and appeared across diseases and genotypes; most of these genes were differentially expressed only in excitatory neurons.

For example, the inventors observed a pan-phenotypic (that is, present in both ALS and FTLD, either sporadic or C9orf72-associated) upregulation of a large number of nuclear-encoded mitochondrial respiratory complex I, III, IV, and V subunit-encoding genes, as well as the mitochondrial membrane transporter ADP/ATP translocase 1 (SLC25A4) and the mitochondrial stability regulator mitoregulin (MTLN). The inventors also observed a primarily upwards, dysregulation of a substantial fraction of ribosomal subunit-encoding genes belonging to both the cytoplasmic (RPL/S) and mitochondrial (MRPL/S) families, the former of which was dramatically higher in excitatory neurons of c9ALS patients compared to the other cohorts.

Of particular note was the pan-phenotypic upregulation of several other genes previously linked to ALS and FTLD, including several heat shock proteins (HSP90AA1, HSP90AB1, HSPA8), Calmodulin-1 (CALM1), and the DEAD-box transcription and splicing regulator DDX24, which were among the most highly upregulated genes and present in nearly every excitatory subtype and phenotype and a few inhibitory subtypes. As the heat shock response is activated to prevent proteins from denaturing and aggregating under stress conditions, the HSF1 pathway and various heat shock proteins have been studied in the context of ALS and FTLD. Indeed Arimoclomol, a co-inducer of the heat shock protein response that may enhance the HSF1 pathway, is the focus of multiple clinical trials after delaying disease progression in mice, including a Phase II/III clinical trial for patients with rapidly progressive ALS caused by SOD1 mutations.

Recent results suggest this drug may have therapeutic benefits for FTLD as well. In addition, CALM1 has been proposed as a potential biomarker of longevity in a SOD1 mouse model, and DDX24 was shown to be differentially expressed in ALS blood.

Neurofilament subunits (including NEFL, NEFM, NEFH, INA) were another notable class of genes driving the common pan-phenotypic signature. Alterations to NEFH, which encodes neurofilament heavy (NHF), are uncommon but found in ALS, and overexpression of NFH leads to motor neuron degeneration in transgenic mice. Neuronal cytoskeleton-associated proteins (including STMN2, TUBB2A, HOOK2) were also commonly upregulated genes in excitatory neurons. STMN2 is highly expressed in the central nervous system, and its expression is increased after neuronal injury. Similar to NEFH, increased STMN2 expression is also observed in other neurodegenerative diseases and has been linked to TDP-43 pathobiology. TDP-43 regulates the splicing of STMN2, and TDP-43 cytoplasmic re-localization leads to a truncated STMN2 mRNA, reduced STMN2 protein levels, and reduced neuronal outgrowth. Even though these changes in cytoskeletal genes were pan-neuronal, their magnitude of upregulation was particularly high in Betz cells for each phenotype (FIG. 3D), suggesting a possible compensatory response against the degradation of axonal integrity, as Betz cells are most affected. In addition, the L3/L5 LR cell type showed a surprisingly similar, and sometimes more severe, degree of dysregulation of these genes, particularly the neurofilament-encoding genes, indicating that the broad dysregulation of long-range projecting cells is not limited to Betz cells. Lastly, a subtype of L5 cells (Ex L5 LRRK1 COL21A1) also showed extensive dysregulation that was particularly prominent in ALS (FIG. 3B).

Inhibitory neurons showed more heterogeneous disease signatures within and across phenotypes, and significantly less dysregulation overall (FIG. 3A, 3B). Nevertheless, interneuronal subtypes also showed upregulation of heat shock proteins and a few of the top-ranking excitatory DEGs, implying that these genes are part of a pan-neuronal disease or stress response. In contrast, all glial and vascular cell types showed little to no overlap in DEGs with either neuronal class or with each other and were overall among the least severely affected cell populations, irrespective of disease.

Across all recovered cell types, downregulated genes showed much more disease-specific patterns, with only a handful of genes appearing across cohorts. However, several genes with links to ALS and FTLD pathobiology were ubiquitously downregulated in neurons within each disease. In both genotypes of ALS, the glutamate receptor subunit-encoding gene GRIN1, and the ALS-linked poly(A) binding protein nuclear 1 (PABPN1) were two of the most downregulated genes in most neuronal subtypes. PABPN1 contains a GCG repeat encoding a polyalanine tract expanded in oculopharyngeal muscular dystrophy (OPMD), plays a role in the regulation of poly(A) site selection for polyadenylation, interacts with TDP-43, MATR3 and hnRNPA1, three genes mutated in both ALS and FTLD, and is a suppressor of TDP-43 toxicity in ALS models. The C9orf72 gene itself was identified as differentially expressed in a small, non-specific subset of excitatory subtypes, and only in C9orf72-associated cohorts, but was only marginally downregulated in these patients.

Since the absolute number of DEGs in a cell population can be biased by various factors (e.g., number of cells, differences in endogenous gene expression across cell types, cellular RNA content), the inventors computed the difference of the mean, transcriptome-wide distance across all cells between diseased and pathologically normal populations for all cell types and disease groups and used this distance as a disease severity score.

This analysis revealed that across cell types, both FTLD cohorts showed a much stronger disease signature than ALS cohorts (FIG. 3B). It also showed that in all disease subgroups, L3/L5 LR and Betz cells exhibited the most drastic transcriptome-wide shift from the pathologically normal profile, with the former showing more dysregulation than the Betz population in three of the four groups (FIG. 3B). This suggests that while ALS- and FTLD-induced transcriptome perturbations may not be unique to Betz cells, these and the ill-defined L3/L5 LR cells, exhibit enhanced vulnerability in both ALS and FTLD relative to other cell types.

The same observation was not made for corticobulbar motor neurons, which showed a degree of transcriptional misregulation comparable to most other non-Betz excitatory neurons. Taken together, our results suggest that, in the primary motor cortex at a molecular level, SCN4B/SV2C+L3/L5 long-range projecting cells (which are FEZF2- and were previously hypothesized to be intratelencephalic projecting) and L5 Betz long-range extratelencephalic-projecting cells are the most affected cell types assessed in this study, for both ALS and FTLD. These cells, and to a lesser extent all other excitatory neurons, display dysregulation of several genes recognized to be genetically or mechanistically linked to ALS and FTLD.

Comparison of Betz cell DEGs across sporadic and C9orf72-associated samples showed strong agreement within genotypes of both ALS (R=0.84) and FTLD (R=0.77), with FTLD showing more heterogeneity. All detected genotype-specific DEGs were uniquely perturbed in either sporadic or C9orf72-associated samples, with none of the highly altered genes showing anti-directional dysregulation in either disease (FIG. 3D). Cross-phenotype comparisons showed weaker, but still respectable correlations, with most uniquely dysregulated or anti-directional perturbations being specific for ALS or FTLD. The marginal differences in transcriptional perturbations observed between genotypes are consistent with the fact that, in the absence of genotype information, sporadic and C9orf72-associated cases of ALS and FTLD are clinically indistinguishable. More broadly, a high-level comparison of DEGs across all cell types revealed that, similar to Betz cells, all other cell types showed modest intra-subtype agreement in transcriptional alterations, even across dissimilar disease groups where few or no other subtype pairs exhibited any degree of similarity.

### Betz Cell Biological Pathway Alterations in ALS and FTLD

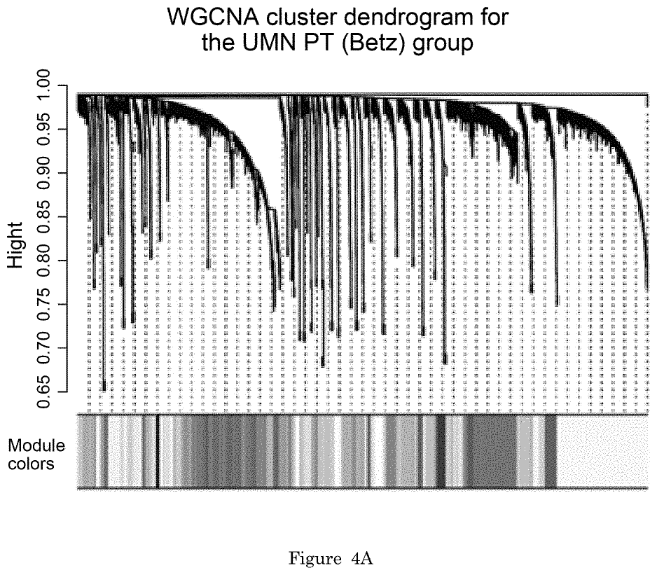

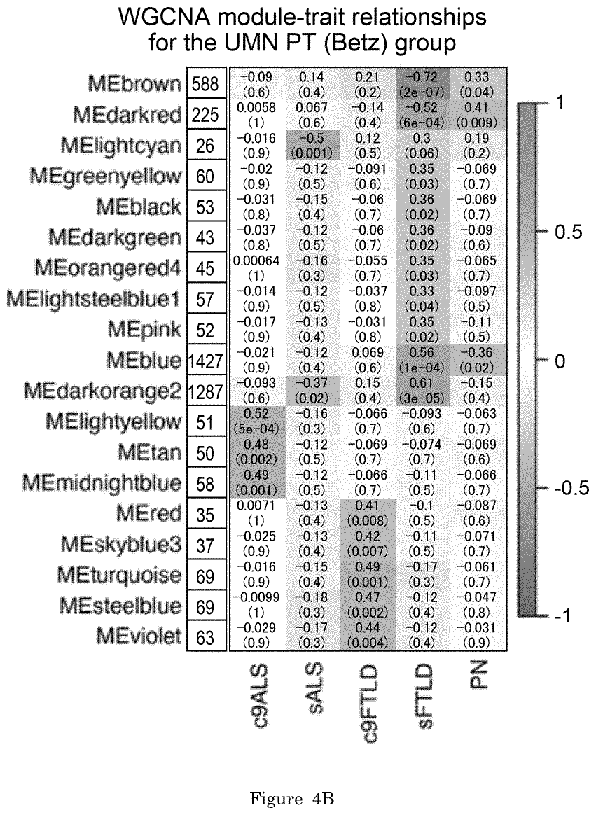

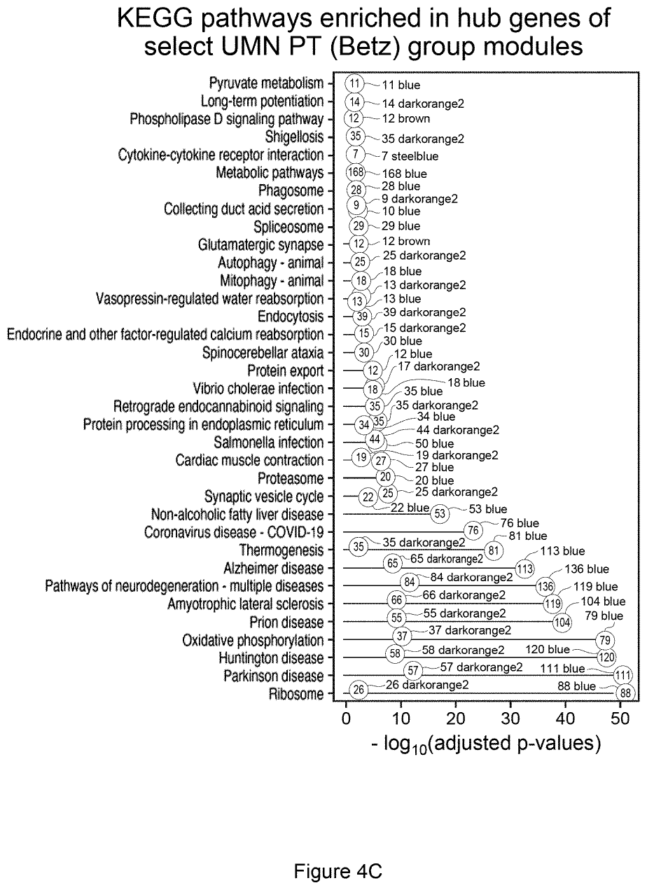

As Betz cells exhibited a large transcriptome-wide shift in both ALS and FTLD and are known to be affected in both diseases 10, the inventors sought to identify trait-specific putative gene regulatory networks that are specifically altered in this cell population. For this purpose, the inventors used weighted gene co-expression network analysis (WGCNA), which summarizes co-expressed gene clusters into “modules” and is widely utilized to identify highly correlated genes via unsupervised clustering Using pseudo-bulk transcriptional data converted from the single-nuclear transcriptional data described above, our analysis revealed 46 modules specific to ALS and FTLD Betz cells (FIG. 4A). Among these, 19 modules significantly correlated with clinical traits (sporadic and C9orf72-associated ALS and FTLD; FIG. 4B). The inventors found that sporadic FTLD had the most correlated modules, followed by c9FTLD, c9ALS, and sALS. Notably, the “darkorange2” module correlated with both sporadic ALS and FTLD groups. Interestingly, all four members of the stathmin family of genes (STMN1, STMN2, STMN3, and STMN4) were identified as top hub genes for this module, having expression values highly correlated with the “darkorange2” module's eigengene values73. Many hub genes of the “darkorange2” module were also known ALS- and FTLD-associated genes, including DCTN1, CHCHD10, SOD1, SQSTM1, VAPB, VCP, UBQLN2, PFN1, and PRNP, a known C9orf72 age-of-onset modifier.

Analysis of hub genes for the 19 modules revealed many shared pathways from both “blue” and “darkorange2” modules (FIG. 4C). The significant “blue” and “darkorange2” pathways were related to stress response, and included terms such as ribosome, oxidative phosphorylation, synaptic vesicle cycle, protein processing in endoplasmic reticulum, and autophagy. Importantly, impaired stress response is indeed a widely recognized pathological mechanism for both ALS and FTLD, and includes oxidative stress, endoplasmic reticulum stress, disruption of major protein clearance pathways such as ubiquitin-proteasome system and autophagy, altered stress granules dynamics, unfolded protein response, and DNA damage/repair response. Also of interest, nuclear pore complex (NPC) and nucleocytoplasmic transport (NCT) defects were common terms for sporadic and C9orf72-associated ALS and FTLD, and hub genes included nuclear pore complex NUP50, nuclear transport receptor TNPO3, and arginine methyltransferase PRMT1. NPC and aberrant NCT have received a great deal of interest in recent years after being first observed in C9orf72-associated diseases and then sporadic cases. The hub genes identified here are also known modifiers of NPC and NCT, and are associated with multiple neurodegenerative diseases including ALS and FTLD, including Nup50 mutations demonstrated to be genetic suppressors of TDP-43 toxicity. Disruption in NPC and NCT affects the localization of multiple proteins involved in the stress response such as ribosomal proteins and stress granule-associated RNA binding proteins. A number of hub genes identified encode proteins interacting with G3BP1 (CAPRIN1, CSDE1, POLR2B, EIF3G, DDX3X, NUFIP2, EEF2), which is known to specifically bind to the Ras-GTPase-activating protein, a key regulator of NCT. Of interest, dysregulated NPCs were shown to be degraded via upregulation of the ESCRT-III/Vps4 Complex in Drosophila models of C9orf72 ALS, and components of the four core subunits of the ESCRT-III complex (CHMP4B, CHMP1A, CHMP5) and of other ESCRT complexes (VPS28 and VPS25) were also “darkorange2” hub genes.

Further inspection of hub genes revealed other interesting regulatory networks that are potentially contributing to ALS and FTLD pathogenesis, such as SRSF3 and SRSF8. SRSF proteins are pre-mRNA splicing factors with multiple functions, including mRNA export from the nucleus. Some SRSF proteins have been found to be sequestered by C9orf72-associated RNA foci (SRSF1; SRSF2), and SRSF1, SRSF3, SRSF7 were shown to have increased binding to the GGGGCC (G4C2) C9orf72 expanded repeat, potentially overriding normal nuclear retention, encouraging nuclear export of repeat-expanded pre-mRNAs, and consequently leading to repeat-associated non-AUG (RAN) translation and dipeptide repeats (DPR) in C9orf72-associated diseases. Other hub genes included the RNA-G4s helicase DDX3X, encoding a protein involved in transcriptional regulation, pre-mRNA splicing, and mRNA export, and part of the DEAD-box protein family characterized by a conserved Asp-Glu-Ala-Asp (DEAD) motif. DDX3X associates with 5′-UTR RNA Gquadruplexes (rG4s)-containing transcripts, which is especially relevant for C9orf72-associated ALS and FTLD, as the G4C2 C9orf72 expanded repeat leads to a repeat-length-dependent accumulation of rG4- containing transcripts95. Notably, DDX3X was shown to directly bind G4C2 RNAs, consequently suppressing RAN translation, DPR production, and aberrant nucleocytoplasmic transport; its helicase activity is essential for such translation repression. EIF2AK2, another hub gene, encodes the protein kinase R (PKR) which plays a key role in mRNA translation, transcriptional control, and regulation of apoptosis, and is regulated by doublestranded RNAs or double-stranded RNA-binding proteins. TIA1, a gene mutated in both ALS and FTLD and a stress granule marker that colocalizes with TDP-43 inclusions in ALS and FTLD, has been demonstrated to be essential for appropriate activation of the PKR-mediated stress response. A direct connection between EIF2AK2 and TDP-43 was demonstrated when induced TDP-43 toxicity in flies upregulated phosphorylation of EIF2AK2. In addition, MARK2, a hub gene in the “lightcyan” module, encodes a protein involved in the stability control of microtubules. MARK2 specifically phosphorylates eIF2a in response to proteotoxic stress and is activated via phosphorylation in ALS. Inhibition of eIF2a-phosphorylation has been demonstrated to mitigate TDP-43 toxicity.

The inventors also observed hub genes involved in N6-methyladenosine (m6A) RNA metabolism such as RBM15B, a component of a regulatory protein complex that regulates m6A “writer”, and ALKBH5, an m6A “eraser” involved in global m6A demethylation. Genes involved in the degradation of m6A-containing mRNAs such as deadenylase CNOT7 and RPP25, a shared component of ribonuclease (RNase) P and RNase mitochondrial RNA-processing (MRP), were also found as hub genes in the “darkorange2” module. These observations indicate potential disruption of the m6A RNA methylome in Betz cells.

Additional WGCNA analysis of the cell type showing the highest transcriptional dysregulation, the L3/L5 LR cells, identified 20 modules significantly correlated with the different clinical traits.

Pathway analysis of hub genes from these modules revealed many overlapping pathways altered in the Betz cells, suggesting disruptions of similar pathways in both cell types.

Together, our findings highlight various ‘hub’ genes and biological pathways with prior association with ALS and FTLD, reinforcing the notion that transcriptional dysregulation can be used not only as a molecular marker of disease, but also as a resource to identify new candidate driver genes. Considering putative driver genes, of particular interest is the VEN/Betz cell enriched alanine-repeat encoding gene POU3F1, which appeared as a top 10% “darkorange2” module hub gene (kME value of 0.87, 97th among 1,287 total hub genes). The observation that POU3F1 is highly enriched in VENs and Betz cells (FIG. 2), that it contains a GCG repetitive sequence similar to PABPN1, and that its dysregulation leads to axonal loss, suggest possible involvement in ALS and FTLD pathobiology.

### POU3F1 Alterations in ALS

As short (8-13 repeats) alanine-encoding GCG expansions in the PABPN1 gene cause OPMD, which is characterized by TDP-43-positive aggregates, it is possible that the 11-alanine repeat encoding hub gene POU3F1 represents an intrinsic vulnerability factor of Betz cells and VENs in ALS and FTLD by similarly influencing TDP-43 aggregation. To investigate whether POU3F1 co-localizes with TDP-43 aggregates in ALS and FTLD, the inventors conducted indirect immunofluorescent staining of ALS and FTLD primary motor cortex postmortem tissue samples (FIG. 5). The inventors found that POU3F1 displayed a broad subcellular distribution in Betz cells in pathologically normal tissue. However, in ALS and FTLD patient tissue, the inventors observed a shift in POU3F1 subcellular localization, which now exhibited a punctate morphology in the cytosol of Betz cells. Strikingly, the inventors found that this altered localization of POU3F1 often co-localized with TDP-43 aggregate puncta in Betz cells (FIG. 5), suggesting that TDP-43 may co-aggregate with this Betz cell-enriched transcription factor and contribute to cell type-specific dysfunction in ALS and FTLD. Taken together, these results suggest that the Betz/VEN-enriched transcription factor POU3F1 is mislocalized, consequently impairing its normal cellular functions in ALS and FTLD patient Betz cells.

### Discussion

The inventors report the existence of at least two distinct classes of Betz cells, as well as a previously unappreciated close molecular similarity between Betz cells of the motor cortex and VENs of the frontal insula and dorsolateral PFC, uncovering a novel link between these vulnerable brain regions and lending further evidence to the notion of an ALS-FTLD pathological spectrum. Further studies will be needed to understand how the two classes of Betz cells differ at a functional level, as well as in the context of ALS and FTLD.

The inventors reveal that, in addition to these Betz cells, a recently identified SCN4B/SV2C+ long-range projecting L3/L5 cell type is the most transcriptionally affected in both ALS and FTLD. In the original description, it was postulated that this cell type's markers represent a shift of expression from preferentially layer 5 in mouse to preferentially layer 3 in human. The authors went on to suggest that this population reflects a unique set of human layer 3 pyramidal neurons that may have human (or primate)-specific long-range intracortical projections. Our studies confirmed that these cells are FEZF2−, but that they otherwise express several layer 5 markers (the inventors thus assign them L3/L5 identity), as well as all three neurofilament triplet genes, supporting the idea that they are indeed long-range projection neurons. Further studies will be needed to better understand the cellular and molecular characteristics of this cell population in the normal human brain, as well as in the context of ALS and FTLD, in which they appear to be even more vulnerable than Betz cells. Despite no widespread reports of cell death in this population, some previous pathological studies have suggested that alterations in ALS motor cortex may in fact begin in some pyramidal cells of layer 3. Interestingly, our motor cortex profiles show that these L3/L5 SCN4B/SV2C+ cells are second to Betz cells in terms of sharing molecular similarity to VENs (FIG. 2B, 2C), further reinforcing that these intrinsic molecular characteristics, when possessed by cortical neurons, confer vulnerability to cell loss or dysregulation in ALS and FTLD.

Our studies also reveal many ALS and FTLD-associated pathways and likely driver genes, including the VEN/Betz-cell enriched gene POU3F1, for which the inventors demonstrated that its encoded protein co-aggregates with TDP-43 in Betz cells of ALS and FTLD brain tissue. POU3F1 has been ascribed various roles in the developing nervous system, including in neuronal fate commitment, motor neuron identity, oligodendrocyte differentiation, and Schwann cell differentiation. Developmental perturbations to POU3F1 activity result in axonal abnormalities, myelination abnormalities, and premature death, with knockout of POU3F1 resulting in a fatal breathing defect. However, POU3F1 expression patterns change during development, and its role in the adult nervous system is poorly understood, as is its role in ALS and FTLD pathobiology. Future studies will be needed to understand the role of POU3F1 and other markers shared exclusively across Betz, VEN, and L3/L5 LR cells that our results suggest may be key factors underlying the differential vulnerability of these cell types in ALS and FTLD pathogenesis.

According to the present invention, ALS can be treated and diagnosed using new methods based on the new targets. Therefore, the present invention is a very useful technology, for example, in the medical field.

The present invention includes a new treatment method, a pharmaceutical composition and a diagnostic method for ALS.

An amyotrophic lateral sclerosis (ALS) treatment method according to an embodiment of the present invention includes administering to an ALS patient an inhibitor for a target A or a promoter for a target B.

The target A is at least one gene selected from a group consisting of genes in Table 1-1 or a protein encoded by the gene.

The inhibitor for the target A is a substance that inhibits expression of the gene, or a substance that inhibits a function of the protein encoded by the gene.

The target B is at least one gene selected from a group consisting of genes in Table 1-2 or a protein encoded by the gene.

The promoter for the target B is a substance that promotes expression of the gene, or a substance that promotes a function of the protein encoded by the gene.

An ALS treatment method according to an embodiment of the present invention includes administering to an ALS patient a substance that promotes or inhibits expression of a gene involved in receptor diffusion trapping or a function of a protein encoded by the gene.

An ALS pharmaceutical composition of the present invention contains an inhibitor for a target A or a promoter for a target B.

The target A is at least one gene selected from a group consisting of genes in Table 1-1 or a protein encoded by the gene.

The inhibitor for the target A is a substance that inhibits expression of the gene, or a substance that inhibits a function of the protein encoded by the gene.

The target B is at least one gene selected from a group consisting of genes in Table 1-2 or a protein encoded by the gene.

The promoter for the target B is a substance that promotes expression of the gene, or a substance that promotes a function of the protein encoded by the gene.

An ALS diagnostic method according to an embodiment of the present invention includes acquiring an expression level of a target A or a target B for a biological sample of a subject.

The target A is at least one gene selected from a group consisting of genes in Table 1-1 or a protein encoded by the gene.

When the expression level of the target A of the subject is higher than an expression level of the target A of a healthy subject, it is determined that the subject is suffering from ALS.

The target B is at least one gene selected from a group consisting of genes in Table 1-2 or a protein encoded by the gene.

When the expression level of the target B of the subject is lower than an expression level of the target B of a healthy subject, it is determined that the subject is suffering from ALS.

The present inventors have found new targets showing correlations with ALS, and thus have accomplished the present invention. According to the present invention, treatment and diagnosis of ALS can be performed based on the new targets.

Obviously, numerous modifications and variations of the present invention are possible in light of the above teachings. It is therefore to be understood that within the scope of the appended claims, the invention may be practiced otherwise than as specifically described herein.

1. A method for treating amyotrophic lateral sclerosis, comprising:
administering an inhibitor for a target A or a promotor for a target B to a patient in need thereof,
wherein the target A is at least one gene selected from the group consisting of genes in Table 1-1 or a protein encoded thereby, the inhibitor for the target A inhibits expression of the gene or a function of the protein encoded by the gene, the target B is at least one gene selected from the group consisting of genes in Table 1-2 or a protein encoded thereby, and the promotor for the target B promotes expression of the gene or a function of the protein encoded by the gene.

2. The method according to claim 1, further comprising:
acquiring an expressed amount of the target A or the target B in a biological sample of the patient,
wherein when the expressed amount of the target A of the patient is higher than an expressed amount of the target A of a healthy subject, the inhibitor for the target A is administered to the patient, and when the expressed amount of the target B of the patient is lower than an expressed amount of the target B of the healthy subject, the promotor for the target B is administered to the patient.

3. The method according to claim 1, wherein the patient has no ALS-related mutation in at least one gene selected from the group consisting of a C9orf72 gene, a SOD1 gene, a TBK1 gene, a TARDBP gene, a FUS gene and a NEK1 gene, the target A is a target A1, the target A1 is at least one gene selected from the group consisting of genes in Table 2-1 or a protein encoded thereby, the target B is a target B1, and the target B1 is at least one gene selected from the group consisting of genes in Table 2-2 or a protein encoded thereby.

4. The method according to claim 3, wherein the patient has no ALS-related mutation in a C9orf72 gene.

5. The method according to claim 1, wherein the patient has an ALS-related mutation in a C9orf72 gene, the target A is a target A2, the target A2 is at least one gene selected from the group consisting of genes in Table 3-1 or a protein encoded thereby, the target B is a target B2, and the target B2 is at least one gene selected from the group consisting of genes in Table 3-2 or a protein encoded thereby.

6. The method according to claim 1, wherein the target A is a target A3, the target A3 is at least one gene selected from the group consisting of genes in Table 4-1 or a protein encoded thereby, the target B is a target B3, and the target B3 is at least one gene selected from the group consisting of genes in Table 4-2 or a protein encoded thereby.

7. The method according to claim 2, wherein the biological sample is a sample collected from a cerebrospinal fluid.

8. The method according to claim 1, wherein the inhibitor for the target A inhibits expression of the gene such that the inhibitor for the target A inhibits transcription of or translation from the gene.

9. The method according to claim 8, wherein the inhibitor for the target A is at least one selected from the group consisting of an interfering nucleic acid, an antisense, and a ribozyme, and vectors for expressing these substances.

10. The method according to claim 1, wherein the inhibitor for the target A inhibits a function of the protein encoded by the gene, and is an antibody, an antigen-binding fragment, or an aptamer, against the protein.

11. The method according to claim 1, wherein the promotor for the target B promotes expression of the gene, and is a vector that expresses the gene.

12. The method according to claim 1, wherein the promotor for the target B promotes a function of the protein encoded by the gene, and is the protein encoded by the gene.

13. A pharmaceutical composition for treating ALS, comprising:
an inhibitor for a target A or a promotor for a target B,
wherein the target A is at least one gene selected from the group consisting of genes in Table 1-1 or a protein encoded thereby, the inhibitor for the target A inhibits expression of the gene or a function of the protein encoded by the gene, the target B is at least one gene selected from the group consisting of genes in Table 1-2 or a protein encoded thereby, and the promotor for the target B promotes expression of the gene or a function of the protein encoded by the gene.

14. The pharmaceutical composition according to claim 13, wherein, the target A is a target A1, the target A1 is at least one gene selected from the group consisting of genes in Table 2-1 or a protein encoded thereby, the target B is a target B1, and the target B1 is at least one gene selected from the group consisting of genes in Table 2-2 or a protein encoded thereby.

15. The pharmaceutical composition according to claim 13, wherein the target A is a target A2, the target A2 is at least one gene selected from the group consisting of genes in Table 3-1 or a protein encoded thereby, the target B is a target B2, and the target B2 is at least one gene selected from the group consisting of genes in Table 3-2 or a protein encoded thereby.

16. The pharmaceutical composition according to claim 13, wherein the target A is a target A3, the target A3 is at least one gene selected from the group consisting of genes in Table 4-1 or a protein encoded thereby, the target B is a target B3, and the target B3 is at least one gene selected from the group consisting of genes in Table 4-2 or a protein encoded thereby.

17. The pharmaceutical composition according to claim 13, wherein the pharmaceutical composition is an injection or an infusion.

18. The pharmaceutical composition according to claim 13, wherein the inhibitor for the target A inhibits expression of the gene such that the inhibitor for the target A inhibits transcription of the gene or translation from the gene.

19. The pharmaceutical composition according to claim 18, wherein the inhibitor for the target A is at least one selected from the group consisting of an interfering nucleic acid, an antisense, and a ribozyme, and vectors for expressing these substances.

20. The pharmaceutical composition according to claim 13, wherein the inhibitor for the target A inhibits a function of the protein encoded by the gene, and is an antibody, an antigen-binding fragment, or an aptamer against the protein.

21. The pharmaceutical composition according to claim 13, wherein the promotor for the target B promotes expression of the gene, and is a vector that expresses the gene.

22. The pharmaceutical composition according to claim 13, wherein the promotor for the target B promotes a function of the protein encoded by the gene, and is the protein encoded by the gene.

23. A method for treating ALS, comprising:
acquiring an expressed amount of a target A or a target B in a biological sample of a subject; and
determining that the subject is in need of treating ALS when the expression amount of the target A of the subject is higher than an expression amount of the target A of a healthy subject and/or when the expression amount of the target B of the subject is lower than an expression amount of the target B of the healthy subject,
wherein the target A is at least one gene selected from the group consisting of genes in Table 1-1 or a protein encoded thereby, and the target B is at least one gene selected from the group consisting of genes in Table 1-2 or a protein encoded thereby.

24. The method according to claim 23, further comprising:
acquiring expression of a target A4 out of the targets A or expression of a target B4 out of the targets B in a biological sample of the subject,
wherein the target A4 is at least one gene selected from the group consisting of genes in Table 5-1 or a protein encoded thereby, when an expressed amount of the target A4 of the subject is higher than an expression amount of the target A4 of the healthy subject, the subject is determined to be of a type having no ALS-related mutation in a C9orf72 gene, the target B4 is at least one gene selected from the group consisting of genes in Table 5-2 or a protein encoded thereby, and when an expressed amount of the target B4 of the subject is lower than an expression amount of the target B4 of the healthy subject, the subject is determined to be of a type having no ALS-related mutation in a C9orf72 gene.

25. The method according to claim 23, further comprising:
acquiring expression of a target A5 out of the targets A or expression of a target B5 out of the targets B is measured in a biological sample of the subject,
wherein the target A5 is at least one gene selected from the group consisting of genes in Table 6-1 or a protein encoded thereby, when an expressed amount of the target A5 of the subject is higher than an expression amount of the target A5 of the healthy subject, the subject is determined to be of a type having an ALS-related mutation in a C9orf72 gene, the target B5 is at least one gene selected from the group consisting of genes in Table 6-2 or a protein encoded thereby, and an expressed amount of the target B5 of the subject is lower than an expression amount of the target B5 of the healthy subject, the subject is determined to be of a type having an ALS-related mutation in a C9orf72 gene.

26. The method according to claim 23, wherein the biological sample is a sample collected from a cerebrospinal fluid.

27. A method for treating amyotrophic lateral sclerosis, comprising:
administering an inhibitor or a promotor for a gene expression or a protein function encoded by a target gene to a patient in need thereof,
wherein the target gene is related to receptor diffusion trapping, the inhibitor inhibits the gene expression or the protein function, and the promotor promotes the gene expression or the protein function.

28. The method according to claim 27, wherein the receptor diffusion trapping is postsynaptic neurotransmitter receptor diffusion trapping or neurotransmitter receptor diffusion trapping.
